(19) United States
(12) Patent Application Publication
(10) Pub. No.: US 2026/0007673 A1
(43) Pub. Date: Jan. 8, 2026

(54) ROCK2 INHIBITORS FOR THE TREATMENT OF VIRAL INFECTIONS

(71) Applicant: GRAVITON BIOSCIENCE BV, Amsterdam (NL)

(72) Inventor: Samuel D. Waksal, New York, NY (US)

(21) Appl. No.: 18/998,657

(22) PCT Filed: Jul. 27, 2023
(86) PCT No.: PCT/EP2023/070941

§ 371 Date: Jan. 27, 2025

Related U.S. Application Data
(60) Provisional application No. 63/392,750, filed on Jul. 27, 2022.

(51) Int. Cl. A61K 31/517; A61K 31/506; A61K 31/706; A61K 31/7105; A61P 31/14

(52) U.S. Cl. CPC A61K 31/517; A61K 31/506; A61K 31/706; A61K 31/7105; A61P 31/14

(57) ABSTRACT

The disclosure provides compositions and methods comprising selective inhibitors of Rho-associated coiled-coil kinase 2 (ROCK2) for use in the treatment of viral infections, particularly coronavirus infections such as SARS-CoV-2, and in the treatment of sequelae resulting from the viral infection, including sequelae resulting from coronavirus infection.

### FIELD

The present disclosure relates to methods and compositions for the treatment of viral infections using Rho-associated coiled-coil kinase (ROCK) inhibitors, and particularly inhibitors of Rho-associated coiled-coil kinase 2 (ROCK2).

### BACKGROUND

Viral infections are common among animals and humans. For example, the COVID-19 pandemic, caused by the SARS-CoV-2 virus, has threatened public health all over the world. Strikingly, this pandemic has resulted in >570 million people infected worldwide, with >6 million deaths by mid-2022.

Coronaviruses are a large family of enveloped viruses with a positive-sense, single-stranded RNA genome and belong to the Coronaviridae family, Nidovirales order. Coronavirus infections are concentrated mainly in the upper respiratory system and gastrointestinal tract, although the lower respiratory system may be involved in more serious infections. According to specific virus and host cell types, the symptoms and pathological damage caused by coronavirus infection may be quite different. Some coronaviruses, including HCoV-NL63, HCoV-229E, and HCoV-0C43, continually circulate in the human population and produce mild symptoms similar to the common cold. Other coronaviruses may cause severe respiratory illness with high morbidity and mortality, including Severe Acute Respiratory Syndrome coronavirus (SARS-CoV-1), Middle East Respiratory Syndrome coronavirus (MERS-CoV), and SARS-CoV-2. MERS-CoV was first reported in Saudi Arabia in 2012 and spread to several other countries. SARS-CoV-1 was first recognized in China in 2002 and led to a worldwide outbreak in 2002 and 2003. Other human coronaviruses include 222E (alpha coronavirus), NL63 (alpha coronavirus), OC43 (beta coronavirus), and HKU1 (beta coronavirus).

Persistent and diverse post-viral symptoms have been described in survivors of coronavirus infection, including survivors of Covid-19, even in those with a mild initial disease course. Currently, no curative treatments are available for post-viral syndromes and therapy is directed at symptom alleviation and coping strategies. Additionally, the economic effect of post-viral syndromes can be substantial, including loss of productivity and employment, and increased need for disability benefits and financial support. There is a substantial and unmet need for therapies to treat coronavirus infections, and to treat the sequelae resulting from coronavirus infection.

Rho-associated coiled-coil kinase (ROCK) is a serine/threonine kinase from the AGC (PKA, PKG, and PKC) kinase family and comprises two isoforms, ROCK1 and ROCK2. The two isoforms are expressed and regulated differently in specific tissues. For example, ROCK1 is ubiquitously expressed at a relatively high level, while ROCK2 is preferentially expressed in certain tissues including heart, brain and skeletal muscle. ROCK is a target of the small GTPase Rho and is involved in diverse cellular activities achieved by phosphorylating downstream effector proteins (MLC, LIMK, ERM, MARCKS, CRMP-2, etc.). Studies have shown that various diseases (e.g., pulmonary fibrosis, cardiac-cerebral vascular disease, neurological disease and cancer etc.) are related to the pathways mediated by ROCK. As such, ROCK has been considered as an important target in the development of novel drugs.

The present disclosure relates to the previous unrecognized and surprising potent anti-viral effects of ROCK2 inhibitors and their use for the treatment of viral infections, including coronavirus infections such as a SARS-CoV-1 infection, a SARS-CoV-2 infection, or a MERS-CoV infection. Additionally, ROCK2 inhibition treats many of the secondary conditions that may be result from the viral infection, including inflammation, fibrosis, and cytokine storm. As a result of treating such secondary conditions, ROCK2 inhibition may also be useful for treating or preventing long COVID.

### SUMMARY

In one aspect, the disclosure provides antiviral compositions comprising a Rho-associated coiled-coil kinase (ROCK2) inhibitor, such as a ROCK2 inhibitor disclosed herein. In one aspect, the disclosure provides methods of treating a subject by administering to the subject a therapeutically effective amount of a ROCK2 inhibitor, such as a ROCK2 inhibitor disclosed herein, or a composition comprising the ROCK2 inhibitor. Such methods and compositions described herein may be useful for the treatment of an individual afflicted with or suspected of being afflicted with a viral infection. In embodiments, the viral infection is a coronavirus infection. In embodiments, the viral infection is a SARS-CoV-1 infection, a SARS-CoV-2 infection, or a MERS-CoV infection. In some embodiments, the viral infection is caused by SARS-CoV-2. In some embodiments, the viral infection is caused by a Delta or an Omicron variant of SARS-CoV-2. In embodiments, the methods and compositions described herein are useful for the treatment and prevention of sequelae resulting from the viral infection, including sequelae resulting from coronavirus infection. In embodiments, the methods and compositions described herein are useful for the treatment and prevention of sequelae resulting from a SARS-CoV-1 infection, a SARS-CoV-2 infection, or a MERS-CoV infection. In some embodiments, the sequelae result from a viral infection is caused by SARS-CoV-2. In some embodiments, the sequelae result from a viral infection is caused by a Delta or an Omicron variant of SARS-CoV-2. In embodiments, the sequelae include one or more of the group consisting of fatigue, dyspnea (difficulty breathing), cough, arthralgia (joint pain), myalgia, headache, chest pain, fever, palpitations, myocardial inflammation, ventricular dysfunction, stroke, pulmonary function abnormalities, pulmonary fibrosis, renal dysfunction rash, alopecia, olfactory and/or gustatory dysfunction, sleep dysregulation, cognitive impairment altered, memory impairment, depression, anxiety, changes in mood and combinations thereof. In embodiments, the sequelae include fibrosis. In some embodiments, the fibrosis is pulmonary fibrosis. As a result of treating one or more sequelae of resulting from a SARS-CoV-2 infection, the ROCK2 selective inhibitor may be useful for treating or preventing long COVID.

In one aspect, the disclosure provides a method of treating a viral infection in a subject in need thereof, comprising administering to the subject a therapeutically effective amount of a ROCK2 inhibitor, such as a ROCK2 selective inhibitor, of the disclosure. In some embodiments, the ROCK2 inhibitor is a ribonucleic acid (RNA). In some embodiments, the ROCK2 inhibitor is an antisense RNA against ROCK2 transcription. Optionally, the RNA is a small interfering RNA (siRNA) or a micro RNA (miRNA). In some embodiments, the ROCK2 inhibitor is a compound having the structure of Formula I to IV, as defined herein, or a pharmaceutically acceptable salt, ester, stereoisomer, polymorph, solvate, N-oxide, or isotopically labeled compound thereof, and particularly (2-(3-(4-((1H-indazol-5-yl)amino)quinazolin-2-yl)phenoxy)-N-isopropylacetamide). In some embodiments the ROCK2 inhibitor has the structure of Compound 1, as defined herein, or a pharmaceutically acceptable salt, ester, stereoisomer, polymorph, solvate, N-oxide, or isotopically labeled compound thereof. In some embodiments, the ROCK2 inhibitor is belumosudil. In some embodiments, the subject is a human subject. In some embodiments, the subject is a veterinary subject.

In some embodiments, the method comprises administering to the subject a therapeutically effective amount of at least one other therapeutic agent. The at least one other therapeutic agent may be another antiviral agent, a corticosteroid, an anti-inflammatory signal transduction modulator, a β2-adrenoreceptor agonist bronchodilator, an anticholinergic, a mucolytic agent, hypertonic saline, or a combination thereof.

In one aspect, the disclosure provides a method of treating a viral infection in a subject in need thereof, comprising administering to the subject a therapeutically effective amount of a ROCK2 inhibitor, such as a ROCK2 selective inhibitor, of the disclosure, and a therapeutically effective amount of at least one other antiviral agent. In some embodiments, the ROCK2 inhibitor is a compound having the structure of Formula I to IV, as defined herein, or a pharmaceutically acceptable salt, ester, stereoisomer, polymorph, solvate, N-oxide, or isotopically labeled compound thereof, and particularly (2-(3-(4-((1H-indazol-5-yl)amino)quinazolin-2-yl)phenoxy)-N-isopropylacetamide). In some embodiments the ROCK2 inhibitor has the structure of Compound 1, as defined herein, or a pharmaceutically acceptable salt, ester, stereoisomer, polymorph, solvate, N-oxide, or isotopically labeled compound thereof. In some embodiments, the ROCK2 inhibitor is belumosudil. The at least one other antiviral agent may be a nucleoside or nucleotide analog, or a pharmaceutically acceptable salt or prodrug thereof. In some embodiments, the subject is a human subject. In some embodiments, the subject is a veterinary subject.

### BRIEF DESCRIPTION OF THE DRAWINGS

FIG. 1 provides the viral titers of SARS-CoV-2 (CPN mL⁻¹) in immortalized human airway epithelial cells (Calu-3) after 24 h and 48 h that were infected with the virus and then treated with Compound 2 (10 μL of 10 μM solution) at 0, 2, 4, and 8 h post-infection in triplicate.

FIG. 2 shows the viral titers of SARS-CoV-2 (CPN mL⁻¹) in Calu-3 cells after 24 h that were infected with the virus and then treated with Compound 2 (10 μL of 10 μM solution) or a control solution (DMSO at PBS) at 0, 2, 4, and 8 h post-infection in triplicate.

FIG. 3 shows the viral titers of SARS-CoV-2 (CPN mL⁻¹) in Calu-3 cells after 48 h that were infected with the virus and then treated with Compound 2 (10 μL of 10 μM solution) or a control solution (DMSO at PBS) at 0, 2, 4, and 8 h post-infection in triplicate.

FIG. 4 Cellular Toxicity: LDH Assay. Calu-3 cells were treated with the listed concentrations of Compound 2 for 2 h at 37° C. followed by challenge with SARS-CoV-2 for 6 h. After washing, the cells were maintained for 48 h and the LDH assay performed.

FIG. 5 Cellular Toxicity: LDH Assay. Calu-3 cells were treated with negative control (DMSO), positive control (Saponin; SAP), or the listed concentrations of Compound 2 for 2 h at 37° C. followed by challenge with SARS-CoV-2 for 6 h. After washing, the cells were maintained for 48 h and the LDH assay performed.

FIG. 6 compares the viral titers of SARS-CoV-2 (CPN mL⁻¹) in Calu-3 cells after 24 h that were infected with the virus and then treated with negative control (DMSO in PBS), positive control (remdesivir, “RDV”), or Compound 2 (10 μM solution) at 0, 1, 2, 4, 8, and 10 h post-infection.

FIG. 7 compares the viral titers of SARS-CoV-2 (CPN mL⁻¹) in Calu-3 cells after 48 h that were infected with the virus and then treated with negative control (DMSO in PBS), positive control (remdesivir, “RDV”), or Compound 2 (10 μM solution) at 0, 1, 2, 4, 8, and 10 h post-infection.

FIGS. 8A-8F provide dose-response graphs, generated from data obtained 24 h after infection, illustrating the viral concentration of SARS-CoV-2 (CPN mL⁻¹) in Calu-3 cells that were infected with the virus and then treated with solutions of negative control (DMSO in PBS) or Compound 2 (1, 5, 25, or 25 μM) at 0 h (FIG. 8A), 1 h (FIG. 8B), 2 h (FIG. 8C), 4 h (FIG. 8D), 8 h (FIG. 8E), and 10 h (FIG. 8F) post-infection.

FIGS. 9A-9F provide dose-response graphs, generated from data obtained 48 h after infection, illustrating the viral concentration of SARS-CoV-2 (CPN mL⁻¹) in Calu-3 cells that were infected with the virus and then treated with solutions of negative control (DMSO in PBS) or Compound 2 (1, 5, 25, or 25 μM) at 0 h (FIG. 9A), 1 h (FIG. 9B), 2 h (FIG. 9C), 4 h (FIG. 9D), 8 h (FIG. 9E), and 10 h (FIG. 9F) post-infection.

FIG. 10 provides the viral concentration of SARS-CoV-2 (CPN mL⁻¹) 48 h after infection in Calu-3 cells treated with negative control (DMSO in PBS), positive control (RDV), or Compound 2 at 0, 1, 2, 4, 8, and 10 h post-infection, replenishing at 24 h with either untreated culture medium (“unmediated medium”) or culture medium containing the positive control or Compound 2 (“mediated medium”).

FIG. 11 compares the viral concentration of SARS-CoV-2 (CPN mL⁻¹) in primary human airway epithelial cells 24 h after infection following treatment with Compound 2 at 5 vs. 25 μM concentrations at 0 h, 1 h, 2 h, 4 h, 8 h, and 10 h.

FIG. 12 compares the viral concentration of SARS-CoV-2 (CPN mL⁻¹) in primary human airway epithelial cells 48 h after infection following treatment with Compound 2 at 5 vs. 25 μM concentrations at 0 h, 1 h, 2 h, 4 h, 8 h, and 10 h.

FIG. 13 provides the number of copies of SARS-CoV-2 virus in the lungs of mice infected with 100 pfu of the virus and treated with vehicle (carboxymethyl cellulose (“CMC”)), Compound 2, or remdesivir, “RDV” following 7, 14, and 21 days of treatment. Groups from left to right: Vehicle, Compound 2 (300 mg/kg), Remdesivir (RDV). Insert: Compound 2 (300 mg/kg) (left), Remdesivir (RDV) (right).

FIG. 14 provides the number of copies of SARS-CoV-2 virus in the lungs of mice infected with 1000 pfu of the virus and treated with vehicle (carboxymethyl cellulose (“CMC”)), Compound 2, or remdesivir, “RDV” following 7, 14, and 21 days of treatment. Groups from left to right: Vehicle, Compound 2 (300 mg/kg), Remdesivir (RDV). Insert: Compound 2 (300 mg/kg) (left), Remdesivir (RDV) (right).

FIGS. 15A and 15B provide the number of copies of SARS-CoV-2 virus (Delta (FIG. 15A) and Omicron (FIG. 15B) variants) in the lungs of mice infected with 100 or 1000 pfu of the virus and treated with vehicle (carboxymethyl cellulose (“CMC”)), Compound 2 (100 mg/kg and 300 mg/kg), or remdesivir, “RDV” following 7, 14, and 21 days of treatment.

FIG. 16 histological images uninfected (“naïve”) or SARS-CoV-2 infected lung tissue obtained 7, 14, and 21 days after infection that were treated with either vehicle (carboxymethyl cellulose (“CMC”)) or Compound 2. The data demonstrate that Compound 2 reduces lung fibrosis in SARS-CoV-2 infected tissue.

FIG. 17 provides the observed relief rate of sustained clinical symptoms for patients suffering from SARS-CoV-2 infection who did not receive treatment with Compound 2 (solid line (Control—placebo)) vs. those who did (long dashes (Ex. Group A —200 mg/day) and short dashes (Ex. Group B—400 mg/day)).

FIG. 18 provides the negative conversion rate of SARS-CoV-2 nucleic acid detection in patients suffering from SARS-CoV-2 infection who did not receive treatment with Compound 2 (solid line (Control—placebo)) vs. those who did (long dashes (Ex. Group A—200 mg/day) and short dashes (Ex. Group B—400 mg/day)).

FIG. 19 compares the observed inflammation, using interleukin 6 (IL-6) as an indicator, in patients suffering from SARS-CoV-2 infection who did not receive treatment with Compound 2 (triangle, solid line (Control—placebo)) vs. those who did (circle, long dashes (Ex. Group A—200 mg/day) and square, short dashes (Ex. Group B—400 mg/day)).

FIG. 20 compares the observed inflammation, using C-reactive protein (CRP) as an indicator, in patients suffering from SARS-CoV-2 infection who did not receive treatment with Compound 2 (triangle, solid line (Control—placebo)) and those who did (circle, long dashes (Ex. Group A—200 mg/day) and square, short dashes (Ex. Group B—400 mg/day)).

### DETAILED DESCRIPTION

The compounds, compositions and methods described herein provide inhibitors of Rho-associated coiled-coil kinase 2 (ROCK2), including selective inhibitors of ROCK2, for use in the treatment of viral infections, particularly coronavirus infections such as a SARS-CoV-1 infection, a SARS-CoV-2 infection (including all variants, such as Delta and Omicron), or a MERS-CoV infection, and in the treatment and prevention of sequelae resulting from the viral infection, including sequelae resulting from coronavirus infection.

### ROCK2 Inhibitors

The compounds for use in the methods and compositions disclosed herein are ROCK inhibitors, and in particular ROCK2 selective inhibitors. The compounds provide excellent inhibitory activity of ROCK (preferably ROCK2), good selectivity (higher selectivity towards ROCK2 as compared with ROCK1), good physicochemical properties (e.g., solubility, physical and/or chemical stability), improved pharmacokinetic properties (e.g., improved bioavailability, proper half-life and duration of action), improved safety (low toxicity and/or less side effects, wide therapeutic window), and the like.

As provided herein, ROCK2 inhibitors have previously unrecognized and surprisingly potent anti-viral effects. Without being bound by theory, ROCK2 inhibitors, such as, for example, an antisense RNA, siRNA, or miRNA against ROCK2 transcription or compounds having the structure of the Formulas I to IV, as defined herein, may interfere with one or more of: (1) pathways used by the virus to enter cells; (2) the cellular cytoskeleton used by the virus as a track for migration and spread; (3) pathways used by the virus to upregulate the energy metabolism of a cell; and (4) pathways used by the virus to spread to other cells. The inhibitors of ROCK2 interfere with viral interaction with cytoskeletons actin filaments, microtubules, and/or intermediate filaments, which are heavily involved in the life cycle and pathological damages caused by viruses, and particularly coronavirus. Additionally, the ROCK2 inhibitors have potent effects against many of the secondary conditions caused by the viral infection, including inflammation, fibrosis, and cytokine and bradykinin storm. Accordingly, the ROCK inhibitors may be useful for treating or preventing long COVID. Long COVID (also called post-COVID) is the appearance of new symptoms or the maintenance of initial symptoms that persist 30 days after initial COVID-19 infection. Long COVID may result in post-COVID fibrosis, which can be seen on lung imaging by CT scan.

According to one aspect, the present disclosure provides methods of treating a subject by administering to the subject a therapeutically effective amount of a ROCK2 inhibitor, such as a ROCK2 selective inhibitor, of the disclosure, or a composition comprising the ROCK2 inhibitor. In some embodiments, the ROCK2 inhibitor is a ribonucleic acid (RNA). In some embodiments, the ROCK2 inhibitor is an antisense RNA against ROCK2 transcription. Optionally, the RNA is a small interfering RNA (siRNA) or a micro RNA (miRNA). In some embodiments, the ROCK2 inhibitor has the structure of Formula I:

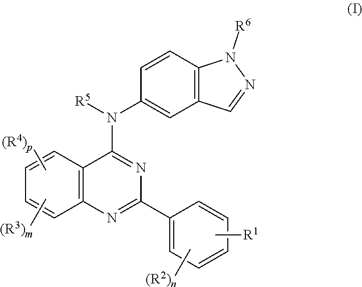

or a pharmaceutically acceptable salt, ester, stereoisomer, polymorph, solvate, N-oxide, or isotopically labeled compound thereof, wherein:
R¹ is selected from the group consisting of —O—(CH₂)ᵣCO₂R¹², —O—(CH₂)_{y}, —C(═O)NR¹³R¹⁴, —O—(CH₂)_{y}, -heteroaryl, —O—(CH₂)_{y}, -cycloalkyl, —O—C(═O)—(CH₂)_{y}, —NR¹³R¹⁴, —O—(CH₂)_{z}—NR¹³R¹⁴, —NH—C(═O)—(CH₂)_{y}, —NR¹³R¹⁴, —NH—C(═O)—X—R¹⁵, and —NH—(CH₂)_{y}, —NR¹³R¹⁴;
R¹² is selected from the group consisting of C₁-C₆ alkyl, —(C₁-C₆ alkyl)-O—(C₁-C₆ alkyl), (C₁-C₆ alkyl)-NR¹⁶R¹⁷, —(C₁-C₆ alkyl)-C(═O)NR¹⁶R¹⁷, —(C₁-C₆ alkyl)-O—(C₁-C₆ alkyl), —O—(C₁-C₆ alkyl), aryl, aralkyl, heteroaryl, C₃-C₇ cycloalkyl, 3-10 membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl;
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
X is absent or selected from a —O, NH, and C₁-C₆ alkyl;
R¹⁵ is selected from the group consisting of heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁵ is selected from (C₁-C₆ alkyl)—O—(C₁-C₆ alkyl), —(C₁-C₆ alkyl)-NR¹⁶R¹⁷, —CO₂R¹⁸, —O—(CH₂)ₓ—CO₂R¹⁸, and —C(═O)NR¹⁶R¹⁷;
R¹⁶ and R¹⁷ are independently selected from the group consisting of H, C₁-C₈ alkyl, C₂-C₈ alkenyl, C₂-C₈ alkynyl, —(C₁-C₆ alkyl)—O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R¹⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR¹⁶R¹⁷, —(C₁-C₆ alkyl)—O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
x is selected from 0 to 6;
y is selected from 0 to 6;
z is selected from 2 to 6;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
R⁴ is selected from H, —(CH₂)ₐ—NR⁴³R⁴⁴, —Y—R⁴², —O—(CH₂)ₐ—CO₂R⁴², —O—(CH₂)ₐ—C(═O)NR⁴³R⁴⁴, —O—(CH₂)ₐ-heteroaryl, —O—(CH₂), -cycloalkyl, —O—C(═O) (CH₂)ₐ—NR⁴³R⁴⁴, —O—(CH₂)ₐ—N⁴³R⁴⁴, —NH—C(═O)—Y—R⁴⁵, —NH—C(═O)—(CH₂)ₐ—NR⁴³R⁴⁴;
R⁴² is selected from the group consisting of C₁-C₆ alkyl, —(C₁-C₆ alkyl)—O—(C₁-C₆ alkyl), —(C₁-C₆ alkyl)-NR⁴⁶R⁴⁷, —(C₁-C₆ alkyl)-C(═O)N⁴⁶R⁴⁷, —(C₁-C₆ alkyl)—O—(C₁-C₆ alkyl)-O—(C₁-C₆ alkyl), each of which may be optionally substituted at one or more carbon atoms by from 1 to 3 substituents independently selected from halo, C₁-C₆ alkoxy, hydroxy, amino, cyano and C₁-C₃ perfluoro alkyl;
R⁴³ and R⁴⁴ are independently selected from the group consisting of H, C₁-C₈ alkyl, C₂-C₈alkenyl, C₂-C₈ alkynyl, —(C₁-C₆ alkyl)—O—(C₁-C₆ alkyl), —(C₁-C₆ alkyl)-NR⁴⁶R⁴⁷, —(C₁-C₆ alkyl)-C(═O)NR⁴⁶R⁴⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁴³ and R⁴⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
Y is absent or selected from O, NH, and C₁-C₆ alkyl;
R⁴⁵ is selected from the group consisting of H, aryl, —(C₁-C₆ alkyl)—O—(C₁-C₆ alkyl), —(C₁-C₆ alkyl)-NR⁴⁶R⁴⁷, —CO₂R⁴⁸, —O—(CH₂)₆—CO₂R⁴⁸, and —C(═O)NR⁴⁶R⁴⁷;
R⁴⁶ and R⁴⁷ independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁴⁶ and R⁴⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁴⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR⁴⁶R⁴⁷, —(C₁-C₆ alkyl)—O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
a is selected from 0 to 6;
b is selected from 0 to 6;
c is selected from 2 to 6;
R⁵ is selected from the group consisting of H, C₁-C₆ alkyl, (CH₂)_{d}—C(═O)NR⁵³R⁵⁴, —C(═O)—(CH₂)_{d}—NR⁵³R⁵⁴, and —C(═O)—X—R⁵⁵;
R⁵³ and R⁵⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁵⁶R⁵⁷, —(C₁-C₆ alkyl)-C(═O)NR⁵⁶R⁵⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁵³ and R⁵⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁵⁵ is selected from the group consisting of H, aryl, —(C₁-C₆ alkyl)-O—(C₁-C₆ alkyl), —(C₁-C₆ alkyl)-NR⁵⁶R⁵⁷, —CO₂R⁵⁸, —O—(CH₂)ₑ—CO₂R⁵⁸, and —C(═O)NR⁵⁶R⁵⁷;
R⁵⁶ and R⁵⁷ independently selected from the group consisting of H, C₁-C₈ alkyl, C₂-C₈ alkenyl, C₂-C₈ alkynyl, —(C₁-C₆ alkyl)—O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁵⁶ and R⁵⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁵⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR⁵⁶R⁵⁷, —(C₁-C₆ alkyl)—O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
d is selected from 0 to 6;
e is selected from 0 to 6;
R⁶ is selected from the group consisting of H, C₁-C₆ alkyl, —(CH₂)ᵣ—C(═O)—NR⁶³R⁶⁴, —C(═O)—(CH₂)ᵣ—NR⁶³R⁶⁴, and —C(═O)—X—R⁶⁵;
R⁶³ and R⁶⁴ are independently selected from the group consisting of H, C₁-C₈alkyl, C₂-C₈ alkenyl, C₂-C₈ alkynyl, —(C₁-C₆ alkyl)—O—(C₁-C₆ alkyl), —(C₁-C₆ alkyl)-NR⁶⁶R⁶⁷, —(C₁-C₆ alkyl)-C(═O)NR⁶⁶R⁶⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁶³ and R⁶⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁶⁵ is selected from the group consisting of H, aryl, —(C₁-C₆ alkyl)-O—(C₁-C₆ alkyl), —(C₁-C₆ alkyl)-NR⁶⁶R⁶⁷, —CO₂R, —O—(CH₂), —CO₂R⁶⁸, and —C(═O)NR⁶⁶R⁶⁷.
R⁶⁶ and R⁶⁷ independently selected from the group consisting of H, C₁-C₅ alkyl, C₂-C₈alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁶⁶ and R⁶⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁶⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR⁶⁶R⁶⁷, —(C₁-C₆ alkyl)—O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
r is selected from 0 to 6;
s is selected from 0 to 6;
n is selected from 0 to 4;
m is selected from 0 to 3; and
p is selected from 0 and 1.

In some embodiments, the ROCK2 inhibitor is a compound having the structure of Formula II:

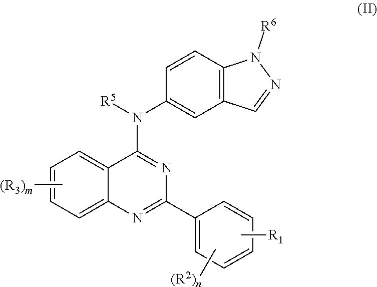

or a pharmaceutically acceptable salt, ester, stereoisomer, polymorph, solvate, N-oxide, or isotopically labeled compound thereof, wherein: R¹, R², R³, R⁵, R⁶, m and n are as defined in Formula I.

In some embodiments, the ROCK2 inhibitor is a compound having the structure of Formula III:

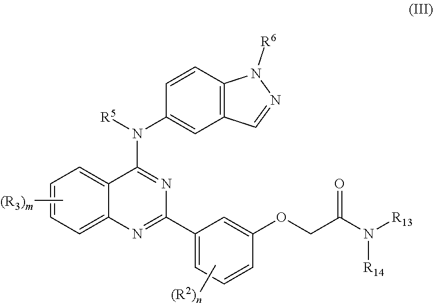

or a pharmaceutically acceptable salt, ester, stereoisomer, polymorph, solvate, N-oxide, or isotopically labeled compound thereof, wherein: R¹³ and R¹⁴ are independently selected from the group consisting of H, C₁-C₈ alkyl, C₂-C₈ alkenyl, C₂-C₈ alkynyl, —(C₁-C₆ alkyl)—O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)—O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
wherein each of R², R³, R⁵, R⁶, m and n are as defined in Formula I.

In some embodiments, the ROCK2 inhibitor is a compound having the structure of Formula IV:

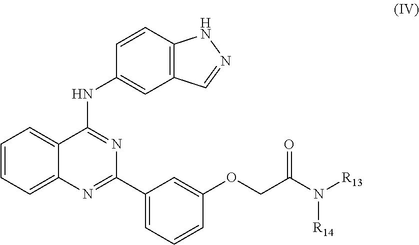

or a pharmaceutically acceptable salt, ester, stereoisomer, polymorph, solvate, N-oxide, or isotopically labeled compound thereof, wherein: R¹³ and R¹⁴ are as defined in Formula III.

In some embodiments, the ROCK2 inhibitor is the compound (2-(3-(4-((1H-indazol-5-yl)amino)quinazolin-2-yl)phenoxy)-N-isopropylacetamide), having the chemical structure of Compound 1:

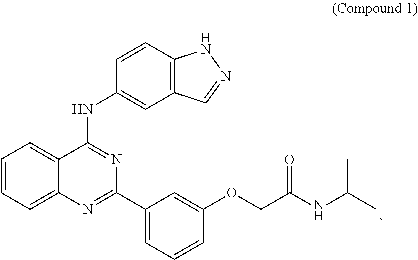

or a pharmaceutically acceptable salt, ester, stereoisomer, polymorph, solvate, N-oxide, or isotopically labeled compound thereof. In some embodiments, the ROCK2 inhibitor is belumosudil.

Compound 1 is a selective inhibitor of Rho-associated coiled-coil kinase 2 (ROCK2) in human cells. Compound 1 or a composition comprising Compound 1 may be used to treat a viral infection.

Compounds of the Formula I to IV, and particularly (2-(3-(4-((1H-indazol-5-yl)amino)quinazolin-2-yl)phenoxy)-N-isopropylacetamide), may be prepared according to the methods disclosed in WO 2014/055996, the contents of which is incorporated herein in its entirety.

As used herein, the term “aliphatic” as used herein refers to a straight chained or branched alkyl, alkenyl or alkynyl. It is understood that alkenyl or alkynyl embodiments need at least two carbon atoms in the aliphatic chain. Aliphatic groups typically contain from 1 (or 2) to 12 carbons, such as from 1 (or 2) to 4 carbons.

As used herein, the term “alkylene” refers to a saturated divalent hydrocarbyl, preferably refers to a saturated divalent hydrocarbyl having 1, 2, 3, 4, 5 or 6 carbon atoms, e.g., methylene (—CH₂—), ethylene (—CH₂CH₂—), propylene or butylene.

As used herein, the term “alkyl” is defined as a linear or branched saturated aliphatic hydrocarbon. In some embodiments, alkyl has 1-12, e.g., 1-6, carbon atoms. For example, as used herein, the term “C₁₋₆ alkyl” refers to a linear or branched group having 1-6 carbon atoms (such as methyl, ethyl, n-propyl, isopropyl, n-butyl, isobutyl, sec-butyl, tert-butyl, n-pentyl, isopentyl, neopentyl, or n-hexyl), which is optionally substituted with one or more (e.g., 1 to 3) suitable substituents such as halogen (in which case the group may be referred to as “haloalkyl”) (e.g., CH₂F, CHF₂, CF₃, CCl₃, C₂F₅, C₂Cl₅, CH₂CF₃, CH₂Cl or CH₂CH₂CF₃, etc.). The term “C₁₋₄ alkyl” refers to a linear or branched aliphatic hydrocarbon chain having 1-4 carbon atoms (i.e., methyl, ethyl, n-propyl, isopropyl, n-butyl, isobutyl, sec-butyl or tert-butyl).

As used herein, the term “alkenyl” refers to a linear or branched monovalent hydrocarbyl having a double bond and 2-6 carbon atoms (“C₂₋₆ alkenyl”). The alkenyl is e.g., vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl and 4-methyl-3-pentenyl. When the compound of the present disclosure contains an alkenylene group, the compound may exist as the pure E (entgegen) form, the pure Z (zusammen) form, or any mixture thereof.

As used herein, the term “alkynyl” refers to a monovalent hydrocarbyl containing one or more triple bond, and preferably having 2, 3, 4, 5 or 6 carbon atoms, e.g., ethynyl or propynyl.

The term “cycloalkyl or cycloalkenyl” refers to a monocyclic or fused or bridged bicyclic carbocyclic ring system that is not aromatic. For example, cycloalkyl, or cycloalkenyl as used herein can be a C₃-C₁₀ monocyclic or fused or bridged C₈-C₁₂ bicyclic carbocyclic ring system that is not aromatic (such as bicyclo[1.1.1]pentyl, bicyclo[2.2.1]heptyl, bicyclo[3.2.1]octyl or bicyclo[5.2.0]nonyl, or decahydronaphthalene etc.)) which is optionally substituted with one or more (e.g., 1 to 3) suitable substituents. Cycloalkenyl rings have one or more units of unsaturation. Preferred cycloalkyl or cycloalkenyl groups include cyclopropyl, cyclobutyl, cyclopentyl, cyclohexyl, cyclohexenyl, cycloheptyl, cycloheptenyl, norbornyl, adamantyl and decalinyl.

As used herein, the terms “cyclic hydrocarbylene”, “cyclic hydrocarbyl” and “hydrocarbon ring” refer to both saturated (i.e., “cycloalkylene” and “cycloalkyl”) or unsaturated (i.e., having one or more double and/or triple bonds in the ring) monocyclic or polycyclic hydrocarbon ring having e.g., 3-10 (suitably having 3-8, and more suitably having 3-6) ring carbon atoms, including but not limited to cyclopropyl(ene) (ring), cyclobutyl(ene) (ring), cyclopentyl(ene) (ring), cyclohexyl(ene) (ring), cycloheptyl(ene) (ring), cyclooctyl(ene) (ring), cyclononyl(ene) (ring), cyclohexenyl(ene) (ring), and the like.

As used herein, the terms “heterocyclyl”, “heterocyclylene” and “heterocycle” refer to a saturated (i.e., heterocycloalkyl) or partially unsaturated (i.e., having one or more double and/or triple bonds in the ring) cyclic group having e.g. 3-10 (suitably having 3-8, and more suitably having 3-6) ring atoms, wherein at least one ring atom is a heteroatom selected from the group consisting of N, O and S, and the remaining ring atoms are C. For example, “3- to 10-membered heterocyclyl(ene)” of “3- to 10-membered heterocycle” refers to saturated or partially unsaturated heterocyclyl(ene) or heterocycle having 2-9 (e.g., 2, 3, 4, 5, 6, 7, 8 or 9) ring carbon atoms and one or more (e.g., 1, 2, 3, or 4) heteroatoms independently selected from the group consisting of N, O and S. Examples of heterocyclylene, heterocyclyl and heterocycle include, but are not limited to oxiranyl(ene), aziridinyl(ene), azetidinyl(ene), oxetanyl(ene), tetrahydrofuranyl(ene), dioxolinyl(ene), pyrrolidinyl(ene), pyrrolidonyl(ene), imidazolidinyl(ene), pyrazolidinyl(ene), pyrrolinyl(ene), tetrahydropyranyl(ene), piperidinyl(ene), morpholinyl(ene), dithianyl(ene), thiomorpholinyl(ene), piperazinyl(ene) or trithianyl(ene). Said group also encompasses a bicyclic system, including a spiro, fused, or bridged system (e.g., 8-azaspiro[4.5]decane, 3,9-diazaspiro[5.5]undecane, 2-azabicyclo[2.2.2]octane, etc.). Heterocyclylene, heterocyclyl and heterocycle may optionally be substituted with one or more (e.g., 1, 2, 3 or 4) suitable substituents.

The term “aryl” as used herein refers to a monocyclic or bicyclic carbocyclic aromatic ring system having a conjugated π electron system. Aryl as used herein includes a (C₆-C₁₂)-aryl-. For example, aryl as used herein can be a C₆-C₁₀ monocyclic or C₈-C₁₂ bicyclic carbocyclic aromatic ring system. In some embodiments, aryl as used herein can be a (C₆-C₁₀)-aryl-. Phenyl (or Ph) is an example of a monocyclic aromatic ring system. Bicyclic aromatic ring systems include 10 systems wherein both rings are aromatic, e.g., naphthyl, and systems wherein only one of the two rings is aromatic, e.g., tetralin. Aryl(ene) or aromatic ring is optionally substituted with one or more (such as 1 to 3) suitable substituents (e.g., halogen, —OH, —CN, —NO₂, and C₁₋₆ alkyl, etc.).

As used herein, the terms “heteroaryl(ene)” and “heteroaromatic ring” refer to a monocyclic, bicyclic or tricyclic aromatic ring system having 5, 6, 8, 9, 10, 11, 12, 13 or 14 ring atoms, particularly 1 or 2 or 3 or 4 or 5 or 6 or 9 or 10 carbon atoms, and containing at least one heteroatom (such as O, N, or S), which can be same to different. Moreover, in each case, it can be benzo-fused. In particular, “heteroaryl(ene)” or “heteroaromatic ring” is selected from the group consisting of thienyl(ene), furyl(ene), pyrrolyl(ene), oxazolyl(ene), thiazolyl(ene), imidazolyl(ene), pyrazolyl(ene), isoxazolyl(ene), isothiazolyl(ene), oxadiazolyl(ene), triazolyl(ene), thiadiazolyl(ene) etc., and benzo derivatives thereof; or pyridinyl(ene), pyridazinyl(ene), pyrimidinyl(ene), pyrazinyl(ene), triazinyl(ene), etc., and benzo derivatives thereof.

As used herein, the term “aralkyl” preferably means aryl or heteroaryl substituted alkyl, wherein aryl, heteroaryl and alkyl are as defined herein. Normally, the aryl group may have 6-14 carbon atoms, the heteroaryl group may have 5-14 ring atoms, and the alkyl group may have 1-6 carbon atoms. Exemplary aralkyl group includes, but is not limited to, benzyl, phenylethyl, phenylpropyl, phenylbutyl.

As used herein, the term “halo” or “halogen” are defined to include F, Cl, Br, or I.

As used herein, the term “nitrogen containing heterocycle” refers to a saturated or unsaturated monocyclic or bicyclic group having 2, 3, 4, 5, 6, 7, 8, 9, 10, 11, 12 or 13 carbon atoms and at least one nitrogen atom in the ring, which may further optionally comprise one or more (e.g., one, two, three or four) ring members selected from the group consisting of N, O, C═O, S, S═O and S(═O)₂. The nitrogen containing heterocycle is attached to the rest of the molecule through the nitrogen atom and any other ring atom in said nitrogen containing heterocycle. The nitrogen containing heterocycle is optionally benzo-fused and is preferably attached to the rest of the molecule through the nitrogen atom in said nitrogen containing heterocycle and any carbon atom in the fused benzene ring.

As used herein, the term “glycoside” refers to any material with a chemical structure comprising a glycosidic bond between a carbohydrate (sugar) molecule and another carbohydrate or a non-carbohydrate (non-sugar) moiety. A glycosidic bond or glycosidic linkage is a type of covalent bond that joins a carbohydrate (sugar) molecule, for example, via its hemiacetal or hemiketal group, to another molecule. The other molecule may or may not be a carbohydrate. The sugar moiety is generally known as the glycone part of a glycoside. The glycone can consist of a single sugar group (monosaccharide) or several sugar groups (oligosaccharide).

As used herein, the term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

As used herein, the term “derivative” refers to a compound that retains the biological activity of the parent compound from which it is derived or is a prodrug for the parent compound. Derivatives may include esters, amides, ethers of the parent compound, obtained by chemically modifying a moiety of the parent compound. For example, a derivative can be a compound in which a hydrogen atom or a certain atomic group is replaced with another atom or atomic group.

The term “substituted” means that one or more (e.g., one, two, three, or four) hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

If a substituent is “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is optionally substituted with one or more of a list of substituents, one or more of the hydrogens on the nitrogen (to the extent there are any) may each be replaced with an independently selected optional substituent.

If substituents are described as being “independently selected” from a group, each substituent is selected independent of the other(s). Each substituent therefore may be identical to or different from the other substituent(s).

As used herein, the term “one or more” means one or more than one (e.g., 2, 3, 4, 5 or 10) as reasonable.

As used herein, unless specified, the point of attachment of a substituent can be from any suitable position of the substituent.

When a bond to a substituent is shown to cross a bond connecting two atoms in a ring, then such substituent may be bonded to any of the ring-forming atoms in that ring that are substitutable.

The compounds for use in the methods provided herein include pharmaceutically acceptable isotopically labeled compounds, which are identical to those of Formulas I to IV, except that one or more atoms are replaced by an atom having the same atomic number, but an atomic mass or mass number different from the atomic mass or mass number which predominates in nature. Examples of isotopes suitable for inclusion in the compounds include, but are not limited to, isotopes of hydrogen, such as ²H, ³H; carbon, such as ¹¹C, ¹³C, and ¹⁴C; chlorine, such as ³⁶C₁; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopically labeled compounds of the present disclosure, for example those incorporating a radioactive isotope, are useful in drug and/or substrate tissue distribution studies (e.g., assays). The radioactive isotopes tritium, i.e., ³H, and carbon-14, i.e., ¹⁴C, are particularly useful for this purpose in view of their ease of incorporation and ready means of detection. Substitution with positron-emitting isotopes, such as ¹¹C, ¹⁸F, ¹⁵O and ¹³N, can be useful in positron emission tomography (PET) studies for examining substrate receptor occupancy. Isotopically labeled compounds of the present disclosure can generally be prepared by processes analogous to those described in the accompanying Schemes and/or in the Examples and Preparations, by using an appropriate isotopically labeled reagent in place of the non-labeled reagent previously employed. Pharmaceutically acceptable solvates in accordance with the disclosure include those wherein the solvent of crystallization may be isotopically substituted, e.g., D₂O, acetone-d₆, or DMSO-d₆.

The term “stereoisomer” refers to isomers with at least one asymmetric center. A compound having one or more (e.g., one, two, three or four) asymmetric centers can give rise to a racemic mixture, single enantiomer, diastereomer mixture and individual diastereomer. Certain individual molecules may exist as geometric isomers (cis/trans). Similarly, the compounds provided herein may exist as a mixture of two or more structurally different forms in rapid equilibrium (generally referred to as tautomer). Typical examples of a tautomer include a keto-enol tautomer, phenol-keto tautomer, nitroso-oxime tautomer, imine-enamine tautomer and the like. It is to be understood that the use of all such isomers and mixtures thereof in any proportion (such as 60%, 65%, 70%, 75%, 80%, 85%, 90%, 95%, 96%, 97%, 98%, and 99%) are encompassed within the scope of the present disclosure.

The present disclosure includes the use of all possible crystalline forms or polymorphs of the compound of the present disclosure, either as a single polymorph, or as a mixture of more than one polymorph, in any ratio.

It also should be understood that certain compounds as provided herein can be used for the treatment in a free from, or where appropriate, in a form of a pharmaceutically acceptable derivative. In the present disclosure, the pharmaceutically acceptable derivative includes, but is not limited to a pharmaceutically acceptable salt, ester, solvate, N-oxide, metabolite, or prodrug, which can directly or indirectly provide the compound of the present disclosure or a metabolite or residue thereof after being administered to a patient in need thereof. Therefore, the compounds provided herein encompass various derivative forms of the compound as mentioned above.

A pharmaceutically acceptable salt of the compounds disclosed herein includes an acid addition salt or a base addition salt. A suitable acid addition salt is formed from an acid which forms a pharmaceutically acceptable salt. Specific non-limiting examples include acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. A suitable base addition salt is formed from a base which forms a pharmaceutically acceptable salt. Specific non-limiting examples include aluminum, arginine, benzathine, calcium, choline, diethylamine, diolamine, glycine, lysine, magnesium, meglumine, olamine, potassium, sodium, tromethamine and zinc salts.

For a review on suitable pharmaceutically acceptable salts, see “Handbook of Pharmaceutical Salts: Properties, Selection, and Use” by Stahl and Wermuth (Wiley-VCH, 2002).

As used herein, the term “ester” refers to those derived from the compounds of the various formulae provided herein, which include physiologically-hydrolyzable esters (which may be hydrolyzed under physiological conditions to release the compounds of the present disclosure in the form of free acids or alcohols).

The compounds for use in the methods of the present disclosure can exist as a solvate (preferably a hydrate), wherein the compound contains a polar solvent, in particular water, methanol or ethanol for example, as a structural element of the crystal lattice of the compound. The amount of the polar solvent, in particular water, may exist in a stoichiometric or non-stoichiometric ratio.

As can be appreciated by a person skilled in the art, not all nitrogen containing heterocycles can form N-oxides since the nitrogen requires an available electron lone-pair for oxidation to the oxide; a person skilled in the art will recognize those nitrogen containing heterocycles which can form N-oxides. A person skilled in the art will also recognize that tertiary amines can form N-oxides. Synthetic methods for the preparation of N-oxides of heterocycles and tertiary amines are well known to a person skilled in the art, and they include the oxidation of heterocycles and tertiary amines with peroxy acids such as peracetic acid and m-chloroperbenzoic acid (mCPBA), hydrogen peroxide, alkyl hydroperoxides such as tert-butyl hydroperoxide, sodium perborate, and dioxiranes such as dimethyldioxirane. These methods for the preparation of N-oxides have been extensively described and reviewed in literatures, see e.g., T. L. Gilchrist, Comprehensive Organic Synthesis, vol. 7, pp 748-750; A. R. Katritzky and A. J. Boulton, Eds., Academic Press; and G W. H. Cheeseman and E. S. G Werstiuk, Advances in Heterocyclic Chemistry, vol. 22, pp 390-392, A. R. Katritzky and A. J. Boulton, Eds., Academic Press.

The compounds described herein may be administered in the form of a prodrug, in which certain derivatives of the compound that may have little or no pharmacological activity itself, can, when administered into or onto the body, be converted into a compound having the desired activity, for example, by hydrolytic cleavage. In general, such prodrug will be a functional derivative of the compound which is readily converted in vivo into the compound with desired therapeutic activity. Further information on the use of the prodrug may be found in “Pro-drugs as Novel Delivery Systems, Vol. 14, ACS Symposium Series (T. Higuchi and V. Stella). The prodrug can, for example, be produced by replacing appropriate functionalities present in the compound of the present disclosure with moieties known to those skilled in the art as “pro-moieties” as described, for example, in “Design of Prodrugs” by H. Bundgaard (Elsevier, 1985).

In some embodiments, the compounds of Formulas I to IV are selective inhibitors of Rho-associated coiled-coil kinase 2 (ROCK2) in human cells. Compounds of the Formulas I to IV, for example as a pharmaceutical composition comprising the compound, are used to treat (i.e., cure or reduce the severity of, etc.) viral infections, particularly coronavirus infections such as SARS-CoV-1, SARS-CoV-2, and MERS-CoV, and to treat or prevent the sequelae resulting from the viral infection, including the coronavirus infection such as SARS-CoV-1, SARS-CoV-2, and MERS-CoV. In some embodiments, the viral infection is a SARS-CoV-1 infection. In some embodiments, the viral infection is a SARS-CoV-2 infection. In some embodiments, the viral infection is caused by a Delta or an Omicron variant of SARS-CoV-2. In some embodiments, the viral infection is a MERS-CoV infection.

Methods of determining kinase inhibition are disclosed herein. For example, kinase activity of an enzyme and the inhibitory capacity of a test compound can be determined by measuring enzyme-specific phosphorylation of a substrate. Commercial assays and kits can be employed. For example, kinase inhibition can be determined using an IMAP® assay (Molecular Devices). This assay method involves the use of a fluorescently tagged peptide substrate. Phosphorylation of the tagged peptide by a kinase of interest promotes binding of the peptide to a trivalent metal-based nanoparticle via the specific, high affinity interaction between the phospho-group and the trivalent metal. Proximity to the nanoparticle results in increased fluorescence polarization. Inhibition of the kinase by a kinase inhibitor prevents phosphorylation of the substrate and thereby limits binding of the fluorescently tagged substrate to the nanoparticle. Such an assay can be compatible with a microwell assay format, allowing simultaneous determination of the IC₅₀ of multiple compounds.

In some embodiments, the ROCK2 inhibitor is not a compound disclosed in U.S. 2019/0276440 or a pharmaceutically acceptable salt, ester, stereoisomer, polymorph, solvate, N-oxide, or isotopically labeled compound of a compound disclosed in U.S. 2019/0276440.

In some embodiments, the ROCK2 inhibitor is not a compound according to Formula V:

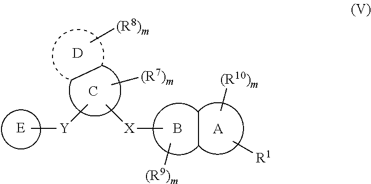

or a pharmaceutically acceptable salt, ester, stereoisomer, polymorph, solvate, N-oxide, or isotopically labeled compound thereof, wherein:
X and Y are each independently selected from the group consisting of a direct bond, C(═O), O, S(═O), and NR;
R is selected from the group consisting of H, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
ring A and ring B are each independently selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O); provided that when ring B is a heterocycle containing a nitrogen atom, ring B is not attached to X via the nitrogen atom;
ring C is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring D is absent, or is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O)
ring E is selected from the group consisting of:

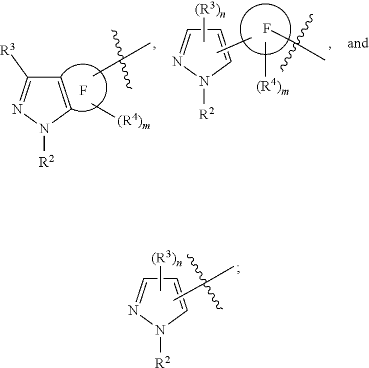

ring F is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
R¹ is selected from the group consisting of H, —NH₂, C₁₋₆ alkyl, C₆₋₁₀ aryl, 5- to 14-membered heteroaryl, N-methylpyrrolidinyl, N-methylpiperidinyl,

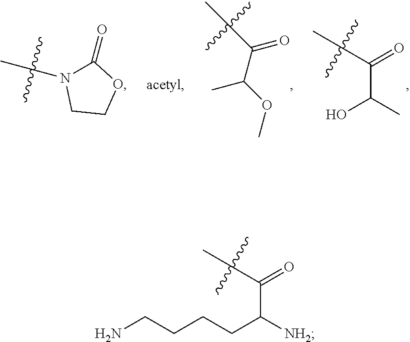

—C(═O)—(C₁₋₆ alkylene)n-CF₃, —C(═O)—(C₁₋₆ alkylene) CN, —C(═O)-(saturated or partially unsaturated C₃₋₁₀ cyclic hydrocarbyl), —NHC(═O)-(saturated or partially unsaturated C₃₋₁₀ cyclic hydrocarbyl), —C(═O)-(saturated or partially unsaturated 3- to 10-membered heterocyclyl), —C(═O)—C₁₋₆ alkylene-(saturated or partially unsaturated 3- to 10-membered heterocyclyl), —C(═O)-(5- to 14-membered heteroaryl), —C(═O)—C₁₋₆ alkylene-NH(C₁₋₆ alkyl), —C(═O)—C₁₋₆ alkylene-N(C₁₋₆ alkyl)₂, N-methylpiperazine substituted acetyl, —S(═O)₂R^{1a}, —P(═O)R^{1a}R^{1b},

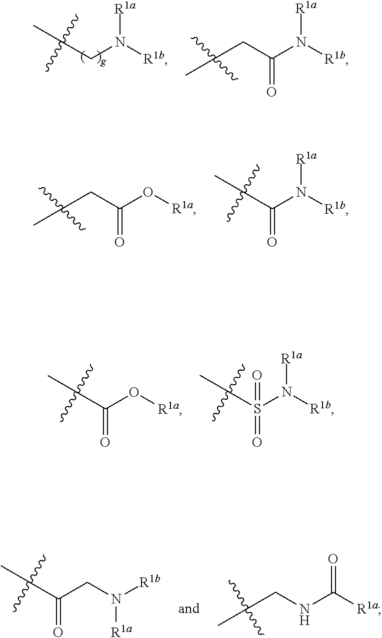

provided that when one of R¹ and R¹⁰ is C¹⁻⁶ alkyl, and the other is H or C³⁻¹⁰ cyclic hydrocarbyl, at least one of X and Y is a direct bond, and ring C is not a 5-membered heteroaromatic ring; when one of R¹ and R¹⁰ is H, and the other is

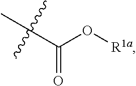

ring C is not a 5-membered heteroaromatic ring; when both R¹ and R¹⁰ are H, ring A contains at least one nitrogen atom, and is not a 5- or 6-membered ring; when one of R¹ and R¹⁰ is H, and the other is

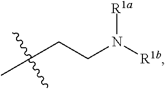

ring C is not a 5-membered heteroaromatic ring; and when one of R¹ and R¹⁰ is H, and the other is H or acetyl, ring D is absent;
R^{1a} and R^{1b} are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, NR⁵—S(═O)₂—R⁶, —NR⁵—C(═O)—NR⁵R⁶, —C₁₋₆ alkylene-NR⁵R⁶, —C₁₋₆ alkylene-OR⁵ and —O—C₁₋₆ alkylene-NR⁵R⁶, provided that when one of R^{1a} and R^{1b} is n-propyl, the other is not H; or R^{1a} and R^{1b} together with the atom to which they are attached form a 3- to 12-membered heterocycle or heteroaromatic ring;
R², R³, R⁴, R⁷, R⁸, R⁹ and R¹⁰, at each occurrence, are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, OC(═O)R⁵, —C(═O)OR⁵, OR⁵, SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, —C₁₋₆ alkylene-NR⁵R⁶, —C₁₋₆ alkylene-O(P═O)(OH)₂ and —O—C₁₋₆ alkylene-NR⁵R⁶;
the above alkyl, alkylene, alkenyl, alkynyl, cyclic hydrocarbyl, hydrocarbon ring, heterocyclyl, heterocycle, aryl, aromatic ring, heteroaryl, heteroaromatic ring and aralkyl, at each occurrence, are each optionally substituted with one or more substituents independently selected from the group consisting of halogen, hydroxyl, oxo, amino, cyano, nitro, C₁₋₆ alkyl, C₂₋₆ alkenyl, C₂₋₆ alkynyl, C₃₋₆ cyclic hydrocarbyl, 3- to 10-membered heterocyclyl, C₆₋₁₀ aryl, 5- to 14-membered heteroaryl,
   C₆₋₁₂ aralkyl, ═N—OR⁵, —C(═NH)NH₂, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, —C₁₋₆ alkylene-NR⁵R⁶ and —O—C₁₋₆ alkylene-NR⁵R⁶, and the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, C₁₋₆ alkyl, C₃₋₆ cyclic hydrocarbyl, 3- to 10-membered heterocyclyl, C₆₋₁₀ aryl, 5- to 14-membered heteroaryl and C₆₋₁₂ aralkyl;
R⁵ and R⁶, at each occurrence, are each independently selected from the group consisting of H, alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3;
n is an integer of 0, 1 or 2;
i is an integer of 0, 1 or 2; and
g is an integer of 0, 1, 2, 3 or 4.

In some embodiments, the ROCK2 inhibitor is not a compound according to any one of Formulae VI to XIII:

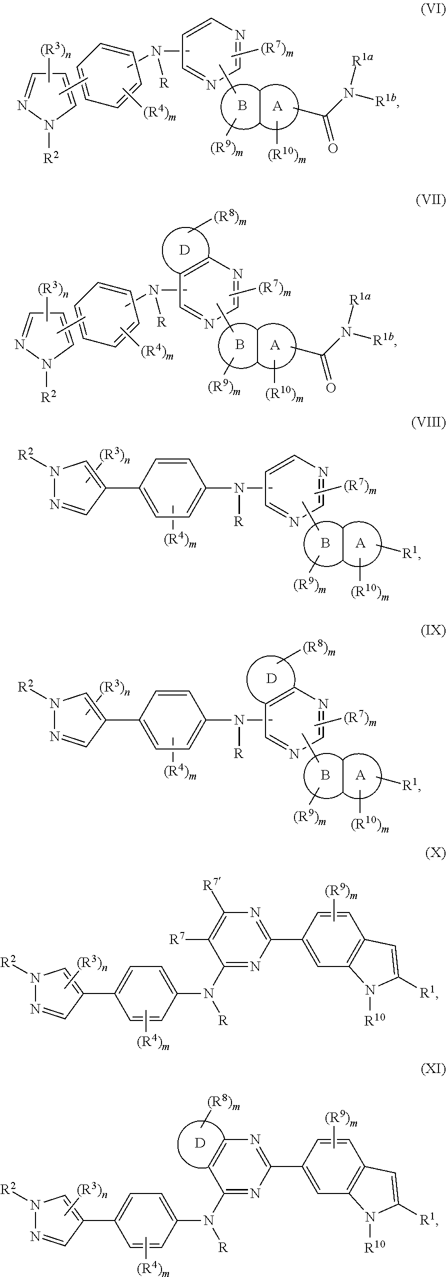

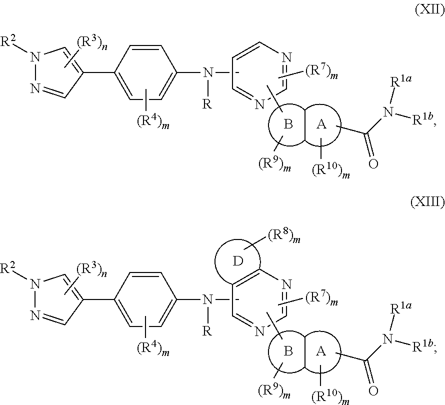

or a pharmaceutically acceptable salt, ester, stereoisomer, polymorph, solvate, N-oxide, or isotopically labeled compound thereof, wherein each of ring A, ring B, ring D, R, R¹, R^{1a}, R^{1b}, R², R³, R⁴, R⁷, R^{7′}, R⁸, R⁹, R¹⁰, n and m are defined as in Formula V above.

In some embodiments, the ROCK2 inhibitor is not a compound according to Formula XIV or XV):

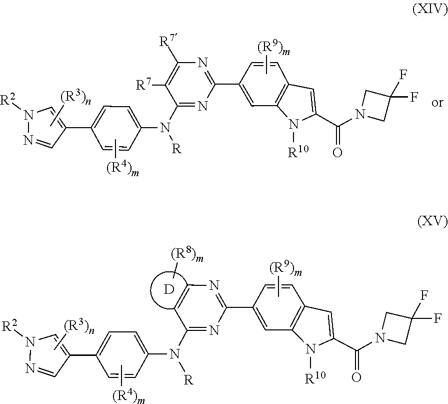

or a pharmaceutically acceptable salt, ester, stereoisomer, polymorph, solvate, N-oxide, or isotopically labeled compound thereof, wherein:
R is selected from the group consisting of H and C₁₋₆ alkyl;
ring D is saturated or partially unsaturated 3- to 10-membered heterocycle, C₆₋₁₀ aryl, or 5- to 10-membered heteroaromatic ring, preferably

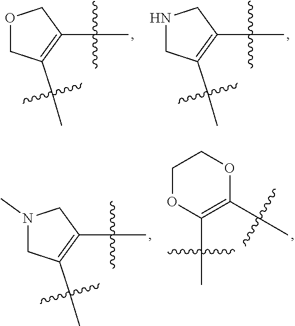

phenyl ring, N-methylpyrrole ring, furan ring or thiophene ring;
R² is selected from the group consisting of H and C₁₋₆ alkyl;
R³, R⁴, R⁷, R^{7′} and R⁸, at each occurrence, are each independently selected from the group consisting of H, halogen, —NH₂, —OH, C₁₋₆ alkyl and —OR⁵;
R⁹ and R¹⁰, at each occurrence, are each independently selected from the group consisting of H, halogen, C₁₋₆ alkyl, C₂₋₆ alkenyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵ and —C₁₋₆ alkylene-O(P═O)(OH)₂;
the above alkyl, alkenyl, cyclic hydrocarbyl, heterocyclyl, aryl, heteroaryl, heteroaromatic ring and aralkyl, at each occurrence, are each optionally substituted with one or more substituents independently selected from the group consisting of halogen, C₁₋₆ alkyl and —OR⁵;
R⁵ and R⁶, at each occurrence, are each independently selected from the group consisting of H, C₁₋₆ alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3; and
n is an integer of 0, 1 or 2.

In some embodiments, the ROCK2 inhibitor is not a compound having the chemical structure of Compound 2:

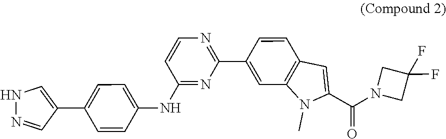 [SMILES: Cn1c(C(=O)N2CC(F)(F)C2)cc2ccc(-c3nccc(Nc4ccc(-c5cn[nH]c5)cc4)n3)cc21]

or a pharmaceutically acceptable salt, ester, stereoisomer, polymorph, solvate, N-oxide, or isotopically labeled compound thereof.

In some embodiments, the ROCK2 inhibitor is not (6-(4-((4-(1H-pyrazol-4-yl)phenyl)amino)pyrimidin-2-yl)-1-methyl-1H-indol-2-yl)(3,3-difluoroazetidin-1-yl)methanone or a pharmaceutically acceptable salt, ester, stereoisomer, polymorph, solvate, N-oxide, or isotopically labeled compound thereof.

In some embodiments, the viral infection is not a coronavirus infection. In some embodiments, the viral infection is not a SARS-CoV-1 infection, a SARS-CoV-2 infection, or a MERS-CoV infection.

In some embodiments, if the viral infection is a coronavirus infection, then the ROCK2 inhibitor is not a compound according to any one Formulae V-XV. In some embodiments, if the viral infection is a coronavirus infection, then the ROCK2 inhibitor is not a compound according to Formula V. In some embodiments, if the viral infection is a coronavirus infection, then the ROCK2 inhibitor is not a compound according to Formula VI. In some embodiments, if the viral infection is a coronavirus infection, then the ROCK2 inhibitor is not a compound according to Formula VII. In some embodiments, if the viral infection is a coronavirus infection, then the ROCK2 inhibitor is not a compound according to Formula VIII. In some embodiments, if the viral infection is a coronavirus infection, then the ROCK2 inhibitor is not a compound according to Formula IX. In some embodiments, if the viral infection is a coronavirus infection, then the ROCK2 inhibitor is not a compound according to Formula X. In some embodiments, if the viral infection is a coronavirus infection, then the ROCK2 inhibitor is not a compound according to Formula XI. In some embodiments, if the viral infection is a coronavirus infection, then the ROCK2 inhibitor is not a compound according to Formula XII. In some embodiments, if the viral infection is a coronavirus infection, then the ROCK2 inhibitor is not a compound according to Formula XIII. In some embodiments, if the viral infection is a coronavirus infection, then the ROCK2 inhibitor is not a compound according to Formula XIV. In some embodiments, if the viral infection is a coronavirus infection, then the ROCK2 inhibitor is not a compound according to Formula XV. In some embodiments, if the viral infection is a coronavirus infection, then the ROCK2 inhibitor is not Compound 2.

In some embodiments, if the viral infection is a SARS-CoV-1 infection, a SARS-CoV-2 infection, or a MERS-CoV infection, then the ROCK2 inhibitor is not a compound according to any one Formulae V-XV. In some embodiments, if the viral infection is a SARS-CoV-1 infection, a SARS-CoV-2 infection, or a MERS-CoV infection, then the ROCK2 inhibitor is not a compound according to Formula V. In some embodiments, if the viral infection is a SARS-CoV-1 infection, a SARS-CoV-2 infection, or a MERS-CoV infection, then the ROCK2 inhibitor is not a compound according to Formula VI. In some embodiments, if the viral infection is a SARS-CoV-1 infection, a SARS-CoV-2 infection, or a MERS-CoV infection, then the ROCK2 inhibitor is not a compound according to Formula VII. In some embodiments, if the viral infection is a SARS-CoV-1 infection, a SARS-CoV-2 infection, or a MERS-CoV infection, then the ROCK2 inhibitor is not a compound according to Formula VIII. In some embodiments, if the viral infection is a SARS-CoV-1 infection, a SARS-CoV-2 infection, or a MERS-CoV infection, then the ROCK2 inhibitor is not a compound according to Formula IX. In some embodiments, if the viral infection is a SARS-CoV-1 infection, a SARS-CoV-2 infection, or a MERS-CoV infection, then the ROCK2 inhibitor is not a compound according to Formula X. In some embodiments, if the viral infection is a SARS-CoV-1 infection, a SARS-CoV-2 infection, or a MERS-CoV infection, then the ROCK2 inhibitor is not a compound according to Formula XI. In some embodiments, if the viral infection is a SARS-CoV-1 infection, a SARS-CoV-2 infection, or a MERS-CoV infection, then the ROCK2 inhibitor is not a compound according to Formula XII. In some embodiments, if the viral infection is a SARS-CoV-1 infection, a SARS-CoV-2 infection, or a MERS-CoV infection, then the ROCK2 inhibitor is not a compound according to Formula XIII. In some embodiments, if the viral infection is a SARS-CoV-1 infection, a SARS-CoV-2 infection, or a MERS-CoV infection, then the ROCK2 inhibitor is not a compound according to Formula XIV. In some embodiments, if the viral infection is a SARS-CoV-1 infection, a SARS-CoV-2 infection, or a MERS-CoV infection, then the ROCK2 inhibitor is not a compound according to Formula XV. In some embodiments, if the viral infection is a SARS-CoV-1 infection, a SARS-CoV-2 infection, or a MERS-CoV infection, then the ROCK2 inhibitor is not Compound 2.

In some embodiments, if the viral infection is a SARS-CoV-2 infection, then the ROCK2 inhibitor is not a compound according to any one Formulae V-XV. In some embodiments, if the viral infection is a SARS-CoV-2 infection, then the ROCK2 inhibitor is not a compound according to Formula V. In some embodiments, if the viral infection is a SARS-CoV-2 infection, then the ROCK2 inhibitor is not a compound according to Formula VI. In some embodiments, if the viral infection is a SARS-CoV-2 infection, then the ROCK2 inhibitor is not a compound according to Formula VII. In some embodiments, if the viral infection is a SARS-CoV-2 infection, then the ROCK2 inhibitor is not a compound according to Formula VIII. In some embodiments, if the viral infection is a SARS-CoV-2 infection, then the ROCK2 inhibitor is not a compound according to Formula IX. In some embodiments, if the viral infection is a SARS-CoV-2 infection, then the ROCK2 inhibitor is not a compound according to Formula X. In some embodiments, if the viral infection is a SARS-CoV-2 infection, then the ROCK2 inhibitor is not a compound according to Formula XI. In some embodiments, if the viral infection is a SARS-CoV-2 infection, then the ROCK2 inhibitor is not a compound according to Formula XII. In some embodiments, if the viral infection is a SARS-CoV-2 infection, then the ROCK2 inhibitor is not a compound according to Formula XIII. In some embodiments, if the viral infection is a SARS-CoV-2 infection, then the ROCK2 inhibitor is not a compound according to Formula XIV. In some embodiments, if the viral infection is a SARS-CoV-2 infection, then the ROCK2 inhibitor is not a compound according to Formula XV. In some embodiments, if the viral infection is a SARS-CoV-2 infection, then the ROCK2 inhibitor is not Compound 2.

### Pharmaceutical Compositions

In one aspect, the present disclosure provides pharmaceutically acceptable compositions for use in the treatment of viral diseases which comprise a therapeutically effective amount of one or more of the ROCK2 inhibitors, including ROCK2 selective inhibitors, disclosed herein, formulated together with one or more pharmaceutically acceptable carriers. In some embodiments, the ROCK2 inhibitor is a ribonucleic acid (RNA). In some embodiments, the ROCK2 inhibitor is an antisense RNA against ROCK2 transcription. Optionally, the RNA is a small interfering RNA (siRNA) or a micro RNA (miRNA). In some embodiments, ROCK2 inhibitor is a compound of Formula I to IV, as defined herein, or a pharmaceutically acceptable salt, ester, stereoisomer, polymorph, solvate, N-oxide, or isotopically labeled compound thereof, and particularly (2-(3-(4-((1H-indazol-5-yl)amino)quinazolin-2-yl)phenoxy)-N-isopropylacetamide). In some embodiments the ROCK2 inhibitor has the structure of Compound 1, as defined herein, or a pharmaceutically acceptable salt, ester, stereoisomer, polymorph, solvate, N-oxide, or isotopically labeled compound thereof. In some embodiments, the ROCK2 inhibitor is belumosudil.

As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a suppository, pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), solvent, or solvent encapsulating material, involved in carrying or transporting the subject compound from one organ, or portion of the body, to another organ, or portion of the body. Each carrier should be compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The ROCK2 inhibitors, including the ROCK2 selective inhibitors, of this disclosure may be formulated with conventional carriers and excipients, which can be selected in accord with ordinary practice. Tablets can contain excipients, glidants, fillers, binders and the like. Aqueous formulations are prepared in sterile form, and when intended for delivery by other than oral administration generally can be isotonic. All formulations can optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like.

While it is possible for the ROCK2 inhibitors, including the ROCK2 selective inhibitors, disclosed herein to be administered alone, it may be preferable to present them as pharmaceutical formulations. The formulations, both for veterinary and for human use, of the disclosure comprise at least one ROCK2 inhibitor, as provided above, together with one or more acceptable carriers therefor and optionally other therapeutic ingredients, particularly those additional therapeutic ingredients as discussed herein.

The formulations include those suitable for the administration routes provided herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the ROCK2 inhibitor with the carrier which constitutes one or more accessory ingredients. In general, the formulations are prepared by uniformly and intimately bringing into association the ROCK2 inhibitor with liquid carriers or finely divided solid carriers or both, and then, if necessary, shaping the product.

Formulations of the present disclosure suitable for oral administration may be presented as discrete units such as capsules, cachets or tablets, each containing a predetermined amount of the ROCK2 inhibitor; as a powder or granules; as a solution or a suspension in an aqueous or non-aqueous liquid; or as an oil-in-water liquid emulsion or a water-in-oil liquid emulsion. The ROCK2 inhibitor may also be administered as a bolus, electuary or paste.

A tablet may be made by compression or molding, optionally with one or more accessory ingredients. Compressed tablets may be prepared by compressing in a suitable machine the ROCK2 inhibitor in a free-flowing form such as a powder or granules, optionally mixed with a binder, lubricant, inert diluent, preservative, surface active or dispersing agent. Molded tablets may be made by molding in a suitable machine a mixture of the powdered ROCK2 inhibitor moistened with an inert liquid diluent. The tablets may optionally be coated or scored and optionally are formulated so as to provide slow or controlled release of the ROCK2 inhibitor therefrom.

For infections of the eye or other external tissues e.g., mouth and skin, the formulations are preferably applied as a topical solution, ointment or cream containing the ROCK2 inhibitor(s). The ROCK2 inhibitor may be present in an amount of, for example, 0.075 to 20% w/w (including ROCK2 inhibitor(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the ROCK2 inhibitors may be employed with either a paraffinic or a water-miscible ointment base. Alternatively, the ROCK2 inhibitors may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the ROCK2 inhibitor through the skin or other affected areas. Examples of such dermal penetration enhancers include dimethyl sulphoxide and related analogs.

The oily phase of the emulsions of this disclosure may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulsifying agents and emulsion stabilizers suitable for use in the formulation of the disclosure include Tween® 60, Span® 80, cetostearyl alcohol, benzyl alcohol, myristyl alcohol, glyceryl mono-stearate and sodium lauryl sulfate. Further emulsifying agents and emulsion stabilizers suitable for use in the formulation of the disclosure include Tween® 80.

The choice of suitable oils or fats for the formulation is based on achieving the desired properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present disclosure comprise a combination according to the disclosure together with one or more pharmaceutically acceptable carriers or excipients and optionally other therapeutic agents. Pharmaceutical formulations containing the ROCK2 inhibitor may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the ROCK2 inhibitor in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the ROCK2 inhibitor is mixed with an inert solid diluent, for example starch, mannitol, calcium phosphate or kaolin, or as soft gelatin capsules wherein the ROCK2 inhibitor is mixed with water or an oil medium, such as peanut oil, liquid paraffin or olive oil.

Aqueous suspensions of the disclosure contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin and Captisol (=Sulfobutyl ether beta-cyclodextrin; SEB-beta-CD).

Oil suspensions may be formulated by suspending the ROCK2 inhibitor in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the disclosure suitable for preparation of an aqueous suspension by the addition of water provide the ROCK2 inhibitor in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of ROCK2 inhibitor that may be combined with the carrier material to produce a single dosage form can vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 g of the ROCK2 inhibitor per milliliter of solution in order that infusion of a suitable volume at a rate of about 30 mL/hr can occur.

Formulations suitable for topical administration to the eye also include eye drops wherein the ROCK2 inhibitor is dissolved or suspended in a suitable carrier, especially an aqueous solvent for the ROCK2 inhibitor. The ROCK2 inhibitor may be present in such formulations in a concentration of 0.5 to 20%, advantageously 0.5 to 10%, and particularly about 1.5% w/w.

Formulations suitable for topical administration in the mouth include lozenges comprising the ROCK2 inhibitor in a flavored basis, usually sucrose and acacia or tragacanth; pastilles comprising the ROCK2 inhibitor in an inert basis such as gelatin and glycerin, or sucrose and acacia; and mouthwashes comprising the ROCK2 inhibitor in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the ROCK2 inhibitor. Formulations suitable for aerosol or dry powder administration may be prepared according to conventional methods and may be delivered with other therapeutic agents such as compounds.

Formulations suitable for vaginal administration may be presented as suppositories, pessaries, tampons, creams, gels, pastes, foams or spray formulations containing in addition to the ROCK2 inhibitor such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the ROCK2 inhibitor.

The disclosure further provides veterinary compositions comprising at least one ROCK2 inhibitor as above defined together with a veterinary carrier therefor.

Veterinary carriers are materials useful for the purpose of administering the composition and may be solid, liquid or gaseous materials which are otherwise inert or acceptable in the veterinary art and are compatible with the ROCK2 inhibitor. These veterinary compositions may be administered orally, parenterally or by any other desired route.

The ROCK2 inhibitors of the disclosure may be used to provide controlled release pharmaceutical formulations containing as active ingredient one or more ROCK2 inhibitors of the disclosure (“controlled release formulations”) in which the release of the ROCK2 inhibitor is controlled and regulated to allow less frequency dosing or to improve the pharmacokinetic or toxicity profile of a given ROCK2 inhibitor.

Combinations with Other Active Agents

The ROCK2 inhibitors, such as ROCK2 selective inhibitors, disclosed herein may be used in combination with at least one additional therapeutic agent. The at least one additional therapeutic agent may be, for example, an antiviral agent, a corticosteroid, an anti-inflammatory signal transduction modulator, a β2-adrenoreceptor agonist bronchodilator, an anticholinergic, a mucolytic agent, hypertonic saline, or a mixture thereof.

The ROCK2 inhibitor may be administered in combination with one or more additional antiviral therapies and/or antiviral agents. The at least one other antiviral agent may be a nucleoside or nucleotide analog, or a pharmaceutically acceptable salt or prodrugs thereof. The antiviral agent may be selected from remdesivir, ribavirin, favipiravir, T-705 monophosphate, T-705 diphosphate, T-705 triphosphate, ST-193, idoxuridine, edoxudine, trifluridine, vidarabine, brivudine, acyclovir, ganciclovir, valaciclovir, cidofovir, valganciclovir, penciclovir, famciclovir, zidovudine, didanosine, zalcitabine, stavudine, abacavir, lamivudine, emtricitabine, tenofovir disoproxil fumarate, tenofovir alafenamide fumarate, adefovir, entecavir, telbivudine, sofosbuvir, and combinations or mixtures thereof.

It is also possible to combine any ROCK2 inhibitor of the disclosure with one or more additional therapeutic agents in a unitary dosage form for simultaneous or sequential administration to a patient. The combination therapy may be administered as a simultaneous or sequential regimen. When administered sequentially, the combination may be administered in two or more administrations.

Co-administration of a ROCK2 inhibitor of the disclosure with one or more other active therapeutic agents generally refers to simultaneous or sequential administration of a ROCK2 inhibitor of the disclosure and one or more other active therapeutic agents, such that therapeutically effective amounts of the ROCK2 inhibitor of the disclosure and one or more other active therapeutic agents are both present in the body of the patient.

Co-administration includes administration of unit dosages of the ROCK2 inhibitors of the disclosure before or after administration of unit dosages of one or more additional therapeutic agents, for example, administration of the ROCK2 inhibitors of the disclosure within seconds, minutes, or hours of the administration of one or more additional therapeutic agents. For example, a unit dose of a ROCK2 inhibitor of the disclosure can be administered first, followed within seconds or minutes by administration of a unit dose of one or more additional therapeutic agents. Alternatively, a unit dose of one or more additional agents can be administered first, followed by administration of a unit dose of a ROCK2 inhibitor of the disclosure within seconds or minutes. In some cases, it may be desirable to administer a unit dose of a ROCK2 inhibitor of the disclosure first, followed, after a period of hours (e.g., 1-12 hours), by administration of a unit dose of one or more additional therapeutic agents. In other cases, it may be desirable to administer a unit dose of one or more additional therapeutic agents first, followed, after a period of hours (e.g., 1-12 hours), by administration of a unit dose of a ROCK2 inhibitor of the disclosure.

The combination therapy may provide “synergy” and “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the active ingredients are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect which is greater than the predicted purely additive effects of the individual active ingredients of the combination.

### Methods of Treating Viral Infections

In one aspect, the disclosure provides methods for treating a viral infection, particularly coronavirus infections. In some embodiments, the method comprises administering one or more ROCK2 inhibitors, including ROCK2 selective inhibitors, of the disclosure to a subject suffering from the viral infection. The method may comprise administering a therapeutically effective amount of the ROCK2 selective inhibitor. In some embodiments, the method comprises administering one or more ROCK2 inhibitors of the disclosure to a subject at risk for the viral infection. The method may comprise administering a prophylactically effective amount of the ROCK2 selective inhibitor.

In one aspect, the disclosure provides methods for the treatment and prevention of sequelae resulting from the viral infection, including sequelae resulting from coronavirus infection. In some embodiments, the method comprises administering one or more ROCK2 inhibitors, including ROCK2 selective inhibitors, of the disclosure to a subject suffering from the viral infection. The method may comprise administering a therapeutically effective amount of the ROCK2 selective inhibitor.

In one aspect, the disclosure provides methods of preventing a viral infection in a subject at risk for the viral infection, including at risk for a coronavirus infection. In some embodiments, the method comprises administering one or more ROCK2 inhibitors, including ROCK2 selective inhibitors, of the disclosure to the subject at risk for the viral infection. The method may comprise administering a prophylactically effective amount of the ROCK2 selective inhibitor.

As used herein, the term “administering” or “administration” of a ROCK2 inhibitor to a subject refers to contacting the ROCK2 inhibitor to the subject or to a cell, tissue, organ, or biological fluid of the subject. Such administration includes any route of introducing or delivering the ROCK2 inhibitor to perform the intended function. Administration can be carried out by any route suitable for the delivery of the ROCK2 inhibitor. Such administration can be carried out using one of a variety of methods known to those skilled in the art. For example, a ROCK2 inhibitor of this disclosure can be administered systemically or locally. Thus, delivery routes can include oral, inhalational, transdermal, intravenous, intramuscular, intraperitoneal, or subcutaneous delivery. Administration includes self-administration and the administration by another. Administering can also be performed, for example, once, a plurality of times, and/or over one or more extended periods. In some embodiments, the administration includes both direct administration (including self-administration) and indirect administration, including the act of prescribing a drug.

As used herein, the terms “treat,” “treating,” and “treatment” refer the administration of a therapeutic agent, such as a ROCK2 inhibitor or a composition containing any such ROCK2 inhibitor disclosed herein, internally or externally to a subject or patient having one or more disease symptoms, or being suspected of having a disease, for which the agent has therapeutic activity. “Treat,” “treating,” and “treatment” refer to therapeutic treatments and/or prophylactic treatments. These terms cover the treatment of a viral infection described herein, in a subject, such as a human, and include: (i) inhibiting viral infection, i.e., arresting its development; (ii) relieving a viral infection, i.e., causing regression of the infection; (iii) slowing progression of the viral infection; and/or (iv) inhibiting, relieving, or slowing progression of one or more symptoms of the viral infection. Treatment and treating refers to the intentional act of physiological intervention that is intended to cure, retard, or ameliorate one or more symptoms associated with the viral infection. If the treatment is administered prior to clinical manifestation of a viral infection or detection of the viral infection, the treatment is considered prophylactic. The alleviation or reduction of a symptom of a viral infection can be assessed by any clinical measurement typically used by physicians or other skilled artisans to assess the severity or progression of that symptom. The terms further refer to a postponement of development of one or more symptoms and/or a reduction in the severity of one or more symptoms associated with the viral infection. The terms further include ameliorating existing uncontrolled or unwanted symptoms, preventing additional symptoms, and ameliorating or preventing the underlying causes of such symptoms. Thus, the terms denote that a beneficial result has been conferred on the subject.

As used herein, the terms “prevent” and “prevention” refer to acting prophylactically prior to a viral infection, to prevent the viral infection from developing, or to minimize the extent of the viral infection, or to slow its course of development.

As used herein, the terms “patient” “subject” “individual” and the like are used interchangeably herein and refer to any animal amenable to the methods described herein. In some embodiments, the patient, subject or individual is a human. In some embodiments, the subject is a veterinary subject. In some embodiments, the subject is a mammal. In some embodiments, the subject is a companion animal. In some embodiments, the subject is livestock. In some embodiments, the subject is a laboratory animal. In some embodiments, the subject is a cat, a dog, a gorilla, a mink, a ferret, a lion, a tiger, a puma, a cougar, a snow leopard, a bat, a vole, a hamster, a pig, a rabbit, a racoon, a shrew, a deer, a rhesus macaque, a cynomolgus macaque, a baboon, a grivet, a common marmoset, a mouse, and a rat. In some embodiments, the subject is a mink.

As used herein, the term “effective amount” or “therapeutically effective amount” with regard to a ROCK2 inhibitor of the disclosure refers to an amount sufficient to confer a therapeutic benefit in a patient after administration, for example, to improve in the subject one or more symptoms of a viral infection, or to delay, reduce, minimize, mitigate, or ameliorate the symptoms of a viral infection relative to an untreated patient. The effective amount may vary depending on the species, age, weight, health of the subject and the nature or severity of the viral infection. Depending on the mode of administration, the effective amount may vary as well. In some cases, multiple doses of the ROCK2 inhibitor are administered to achieve the effective amount for the therapeutic benefit intended. A therapeutically effective amount is also one in which any toxic or detrimental effects of the antibody are outweighed by the therapeutically beneficial effects. It is routine in the art for the skilled artisan to determine a therapeutically effective amount of an antibody disclosed herein based on these factors. A “prophylactically effective amount” refers to an amount effective, at dosages and for periods of time necessary, to achieve the desired prophylactic result. Typically, since a prophylactic dose is used in subjects prior to a viral infection or at an earlier stage of the viral infection, the prophylactically effective amount may be less than the therapeutically effective amount.

In embodiments, the viral infection is caused by a virus selected from the group consisting of SARS-CoV-1, SARS-CoV-2, MERS-CoV, Yellow Fever, Eastern Equine Encephalitis virus, Human Immunodeficiency virus (HIV), African Swine Fever viruses, Arbovirus, Adenoviridae, Arenaviridae, Arterivirus, Astroviridae, Baculoviridae, Bimaviridae, Bunyaviridae, Caliciviridae, Caulimoviridae, Circoviridae, Coronaviridae, Cystoviridae, EBV, Deltaviridae, Filviridae, Filoviridae, Flaviviridae, Iridoviridae, Mononegavirus, Myoviridae, Papilloma virus, Papovaviridae, Paramyxoviridae, Prions, Parvoviridae, Phycodnaviridae, Poxviridae, Potyviridae, Reoviridae, Retroviridae, Rhabdoviridae, Tectiviridae, Togaviridae, pox, papilloma, influenza, sendai virus (SeV), sindbis virus (SINV), vaccinia viruses, West Nile, Hanta, viruses which cause the common cold, and any combination thereof. In embodiments, the viral infection is caused by a virus selected from the group consisting of Yellow Fever, Eastern Equine Encephalitis virus, Human Immunodeficiency virus (HIV), African Swine Fever viruses, Arbovirus, Adenoviridae, Arenaviridae, Arterivirus, Astroviridae, Baculoviridae, Bimaviridae, Bunyaviridae, Caliciviridae, Caulimoviridae, Circoviridae, Coronaviridae, Cystoviridae, EBV, Deltaviridae, Filviridae, Filoviridae, Flaviviridae, Iridoviridae, Mononegavirus, Myoviridae, Papilloma virus, Papovaviridae, Paramyxoviridae, Prions, Parvoviridae, Phycodnaviridae, Poxviridae, Potyviridae, Reoviridae, Retroviridae, Rhabdoviridae, Tectiviridae, Togaviridae, pox, papilloma, influenza, sendai virus (SeV), sindbis virus (SINV), vaccinia viruses, West Nile, Hanta, viruses which cause the common cold, and any combination thereof. In embodiments, the viral infection is caused by a coronavirus, such as SARS-CoV-1, SARS-CoV-2 and MERS-CoV. In embodiments, the viral infection is caused by SARS-CoV-1. In embodiments, the viral infection is caused by SARS-CoV-2. In some embodiments, the viral infection is caused by a Delta or an Omicron variant of SARS-CoV-2. In embodiments, the viral infection is caused by MERS-CoV.

In embodiments, the disclosure provides a method for treating or preventing one or more sequelae of COVID-19, comprising administering to a subject in need thereof a therapeutically effective amount of a ROCK2 inhibitor, including a ROCK2 selective inhibitor, of the disclosure. In some embodiments the ROCK2 inhibitor is an RNA, such as an antisense RNA, a siRNA or a miRNA. In some embodiments, the ROCK2 inhibitor is a compound of Formulas I to IV, as defined herein, or a pharmaceutically acceptable salt, ester, stereoisomer, polymorph, solvate, N-oxide, or isotopically labeled compound thereof. In embodiments, the disclosure provides a method for treating or preventing one or more sequelae of COVID-19, comprising administering to a subject in need thereof a therapeutically effective amount of Compound 1, as defined herein, or a pharmaceutically acceptable salt, ester, stereoisomer, polymorph, solvate, N-oxide, or isotopically labeled compound thereof. In some embodiments, the disclosure provides a method for treating or preventing one or more sequelae of COVID-19, comprising administering to a subject in need thereof a therapeutically effective amount of belumosudil.

Sequelae of a coronavirus infection, and in particular SARS-CoV-1, SARS-CoV-2 (including all variants, such as Delta and Omicron) and MERS-CoV, may result from one or more phenomena including organ damage from the acute infection phase, manifestations of a persistent hyperinflammatory state, ongoing viral activity associated with a host viral reservoir, or an inadequate antibody response. Sequelae of COVID-19 include, but are not limited to, fatigue, dyspnea (difficulty breathing), cough, arthralgia (joint pain), and chest pain. Additional sequelae include cognitive impairment, depression, myalgia, headache, fever, and palpitations. Sequelae of COVID-19 may be cardiovascular (e.g., myocardial inflammation, ventricular dysfunction, stroke), respiratory (e.g., pulmonary function abnormalities, fibrosis), renal (e.g., acute kidney injury), dermatologic (e.g., rash, alopecia), neurological (e.g., olfactory and gustatory dysfunction, sleep dysregulation, altered cognition, memory impairment), and/or psychiatric (e.g., depression, anxiety, changes in mood). As a result of treating one or more sequelae of COVID-19, the ROCK2 selective inhibitor may be useful for treating or preventing long COVID.

Without being bound by theory, a ROCK2 inhibitor, such as, for example, a ROCK2 selective inhibitor, including antisense RNA, siRNA or miRNA molecules and compounds of the Formulas I to IV and Compound 1, may block: (1) pathways used by the virus to enter cells; (2) the cellular cytoskeleton used by the virus for migration and spread; (3) pathways used by the virus to upregulate the energy metabolism of a cell; and (4) pathways used by the virus to spread to other cells.

The ROCK2 inhibitors, including ROCK2 selective inhibitors, provided herein may interfere with the interaction of the virus with the cytoskeleton of the host cell, and thereby inhibit the entry, replication, and/or spread of the virus. The cytoskeleton is an intricate network in eukaryotic cells, which comprises three major types of cytoskeletal polymers including actin filaments, microtubules, and intermediate filaments, allowing cells to perform multiple functions in a united way, such as connecting to the external environment, coordinating forces to move and change shapes, transporting vesicles through the cytoplasm, and spatially organizing the contents. Most viruses hijack one or more aspects of the cytoskeleton network to facilitate their own infection. The viral interaction with host cell actin filaments, microtubules, and intermediate filaments play the important roles in the life cycle of viruses, and particularly of coronaviruses.

The ROCK2 inhibitors, including ROCK2 selective inhibitors, provided herein may interfere with the entry of a virus into its target cell. After binding to the target cell, viruses may migrate to favorable sites for entry. As the virus reaches the entry site, actin filaments have been observed to retract and concentrate around the plasma membrane. Pharmacological stabilization of the actin cortex may interfere with the viral penetration of the host cells. Viruses also take advantage of cytoskeleton-regulating signaling pathways as part of their infection processes.

Coronavirus may utilize the three cytoskeleton networks to complete viral transport process. Transport from/to the cell periphery for short-range route is mediated by actin and its motor proteins like myosin, while long-range transport is mediated by microtubules and the motor proteins dynein and kinesin. After entering host cell, coronavirus-containing vesicles run along microtubules to move from the plasma membrane toward replication sites.

SARS-CoV-2-infected cells can fuse with neighboring cells to form actin-regulated syncytia. During the infection stage, SARS-CoV-2 surfs along filopodia on the host membrane to the cell entry sites and uses intermediate filament proteins to assist and utilize angiotensin converting enzyme-2 (ACE2) receptors to enter target cells. In a mouse SARS model, the SARS-CoV spike protein bound ACE2 receptors and reduced ACE2 expression leading to severe lung injury. By binding to ACE2 receptors to gain cell entry, SARS-CoV-2 effectively blocks ACE2 activity and the conversion of AT-II to Angiotensin, leading to vasoconstriction, increased vascular permeability, adverse myocardial remodeling, and acute lung injury. The downregulation of ACE2 and interruption of the normal feedback loop can also lead to a life-threatening cytokine storm as commonly observed in moderate to severe cases of COVID-19. An excessive inflammatory response to SARS-CoV-2 represents the main cause of disease severity and death in COVID-19 patients.

When ROCK2 is inhibited, ACE2 activity is restored, AT-II conversion continues, and inflammatory cytokines (i.e., IL-1β, IL-6, and TNF-alpha), TGF-β1, and pro-fibrotic markers (i.e., COL1A1, COL3A1, alpha-SMA, fibronectin, CTGF, and PDGF-B) are downregulated. ROCK inhibition also can play a role in preventing viral spreading between host cells, by inhibiting Rho GTPase mediated cytoskeletal reorganization including movement, migration, survival, cell death, and the formation of and viral syncytia.

Additionally, the neurodevelopmental disorders and respiratory tract damage caused by coronaviruses are, at least in part, microtubule dependent. Structural damage to the respiratory epithelium and abnormal ciliary function are typical pathologic symptoms of coronavirus infection. Cilia is a composite structure present on the cell surface comprised of microtubules. Coronaviruses that cause severe respiratory damage may do so through cilia loss in the upper respiratory tract and lung. Further, disruption of microtubules may be related to neurodegenerative diseases.

Based on its multifaceted modes of action, ROCK2 inhibitors, including ROCK2 selective inhibitors, of the disclosure, such as antisense RNA, siRNA or miRNA of ROCK2 transcripts and compounds of Formulas I to IV and Compound 1 as defined herein, inhibit or prevent viral cell entry and inhibit viral spread, for example by inhibiting the syncytia. Secondarily, the ROCK2 inhibition also mitigates the overactive immune response known as “cytokine storm,” as well as the fibrotic changes in the vascular, cardiac, and lung tissues which have resulted in long-term sequalae for some COVID-19 patients.

In some embodiments, the ROCK2 inhibitor: (a) inhibits viral entry of the viral infection, and/or (b) modulates the viral entry of the viral infection.

In some embodiments, the ROCK2 inhibitor: (a) inhibits viral spread of the viral infection, and/or (b) modulates the viral spread of the viral infection.

In some embodiments, the ROCK2 inhibitor targets a cellular target of the subject. Because the ROCK2 inhibitor interacts with a cellular target of the host, rather than with a viral target, the development of drug resistance to the effects of the ROCK2 inhibitor in the virus may be slowed, or substantially non-existent, when compared with other antiviral agents or compositions. In some embodiments, the use of the ROCK2 inhibitor for the treatment of viral disease is characterized by the absence of drug resistance.

In some embodiments, the ROCK2 inhibitor inhibits stress fiber formation, and/or modulates the stress fiber formation.

In some embodiments, the ROCK2 inhibitor inhibits actin filament dynamics, and/or modulates the actin filament dynamics.

In some embodiments, the ROCK2 inhibitor inhibits the mammalian target of rapamycin (mTOR) metabolic pathway, and/or modulates the mTOR metabolic pathway.

In some embodiments, the ROCK2 inhibitor inhibits the AKT (serine/threonine protein kinase B) metabolic pathway, and/or modulates the AKT metabolic pathway.

In some embodiments, the ROCK2 inhibitor inhibits fusogenic pathway, and/or modulates the fusogenic pathway.

In some embodiments, the subject is a human subject. In some embodiments, the subject is a veterinary subject. In some embodiments, the human subject is an adult patient. In embodiments, the human subject has, or is at risk of having, fibrosis (including post-COVID fibrosis) or scarring on lungs. In some embodiments, the human subject is a pediatric patient. In embodiments, the human subject has, or is at risk of developing, Kawasaki disease or fibrosis or scarring on the lungs.

In embodiments, the ROCK2 inhibitor at least partially reverses and/or inhibits the level of fibrosis, at least partially inhibits the over-deposition of extracellular matrix in the lungs or improves blood supply to the lungs. In some embodiments, the ROCK2 selective inhibitor at least partially reverses and/or inhibits the level of post-COVID fibrosis. In some embodiments, the administration of the ROCK2 inhibitor results in at least one of the following: (a) at least 5% reduction of lung edema; (b) at least 5% reduction of lung pathology severity scores associated with lung fibrosis; (c) at least 5% reduction of expression of pro-inflammatory proteins, or (d) at least 5% reduction of expression of fibrogenic proteins.

In some embodiments, the subject experiences a decline of forced vital capacity of: (a) less than 10%; (b) less than 9%; (c) less than 8%; (d) less than 7%; (e) less than 6%; (f) less than 5%; (g) less than 4%; (h) less than 3%; (i) less than 2%; or (j) less than 1%; following administration of the ROCK2 inhibitor to the subject for at least 2 weeks. In some embodiments, the subject experiences no decline of forced vital capacity following administration of the ROCK2 inhibitor to the subject for at least 2 weeks. In some embodiments, the subject experiences an increase of forced vital capacity of: (a) at least 0.5%; (b) at least 1%; (c) at least 1.5%; (d) at least 2%; (e) at least 2.5%; (f) at least 3%; (g) at least 3.5%; (h) at least 4%; (i) at least 4.5%; or (j) at least 5%; following administration of the ROCK2 inhibitor to the subject for at least 2 weeks.

In some embodiments, the subject experiences a decrease of occurrence of coronary artery lesions at one month of illness of: (a) at least 5%; (b) at least 10%; (c) at least 15%, (d) at least 20%; (e) at least 25%; (f) at least 30%; (g) at least 35%; or (h) at least 40%; following administration of the ROCK2 inhibitor to the subject for at least 2 weeks. In some embodiments, the ROCK2 inhibitor at least partially reverses or reduces vasculitis syndrome, or at least partially reverses or reduces rash, redness to eyes, lips, or tongue, swelling of hands or feet, redness to hands or feet, or neck swelling. In some embodiments, the ROCK2 inhibitor rebalances an immune response in the subject.

In one aspect, the present disclosure provides a method of treating a patient suffering from a viral disease comprising administering to a patient in need of such treatment a therapeutically effective amount of a ROCK2 inhibitor, such as a ROCK2 selective inhibitor of the disclosure. In some embodiments, the ROCK2 inhibitor is an RNA. In some embodiments, the ROCK2 inhibitor is an antisense RNA against ROCK2 transcription. Optionally, the RNA is a small interfering RNA (siRNA) or a micro RNA (miRNA). In some embodiments, the ROCK2 inhibitor is a compound having the structure of Formula I to IV, as defined herein, or a pharmaceutically acceptable salt, ester, stereoisomer, polymorph, solvate, N-oxide, or isotopically labeled compound thereof, and particularly (2-(3-(4-((1H-indazol-5-yl)amino)quinazolin-2-yl)phenoxy)-N-isopropylacetamide). In some embodiments the ROCK2 inhibitor has the structure of Compound 1, as defined herein, or a pharmaceutically acceptable salt, ester, stereoisomer, polymorph, solvate, N-oxide, or isotopically labeled compound thereof. In some embodiments, the ROCK2 inhibitor is belumosudil.

One or more ROCK2 inhibitors, including ROCK2 selective inhibitors, of the disclosure are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It can be appreciated that the preferred route may vary with for example the condition of the recipient. An advantage of the compounds of this disclosure is that they are orally bioavailable and can be dosed orally.

Effective dose of the compounds of the disclosure depends at least on the nature of the condition being treated, toxicity, whether the compound is being used prophylactically or against an active viral infection, the method of delivery, and the pharmaceutical formulation, and can be determined by the clinician using conventional dose escalation studies. In embodiments, doses of the compounds of the disclosure range from about 0.1 to about 50 mg/kg body weight per day. The daily dose for adult human may range from 1 mg to 1000 mg, for example between about 5 mg and about 800 mg or between about 50 mg and 500 mg and may take the form of single or multiple doses per day. In embodiments, the daily dose of the ROCK2 inhibitor to treat the viral infection or treat or prevent one or more sequelae due to the infection is about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg, which may be administered as a single daily dose, or split between two, three or more daily administrations. In embodiments, the dose is about 400 mg per day BID.

Methods of Treating Kawasaki Disease/Symptoms Associated with Virus Infection

Among pediatric patients suffering from COVID-19, some display pediatric multisystem inflammatory Syndrome (PMIS), or pediatric inflammatory multisystem syndrome temporally associated with SARS-CoV-2 (PIMS-TS), which resembles an inflammatory illness called Kawasaki disease. As used herein, the term “Kawasaki disease” generally refers to an inflammatory disease that causes vasculitis syndrome or inflammation of the blood vessels, sometimes swollen throughout the body, including Kawasaki-like disease such as PMIS or PIMS-TS. Symptoms of Kawasaki disease may include a persistent high fever (over 101° F.) for at least four days in addition to rash, redness to eyes, lips/tongue, swelling and redness to hands/feet and neck swelling, etc. As used herein, the term “Kawasaki disease shock syndrome” or KDSS refers to Kawasaki disease patients who present more than 20% decrease in systolic blood pressure compared to healthy subjects of the same age, or to those patients who show peripheral blood circulation perfusion disorder. KDSS can be due to a high level of circulating inflammatory factors.

Among the pediatric patients of Kawasaki associated with COVID-19, PMIS or PIMS-TS, there may be an increase in inflammatory markers including, for example, neutrophil-predominant leukocytosis, C-reactive protein (CRP), procalcitonin (PCT) and IL-6. The development of Kawasaki or PMIS associated with COVID-19 can be caused by a post-viral immunological reaction, such as, for example, antibody or immune-complex mediated reactions.

As a form of systemic vasculitis, Kawasaki disease may involve small to medium-sized blood vessels. The most severe complication or sequela may be the formation of coronary artery lesions (CAL), such as myocardial infarction, coronary artery fistula, coronary artery dilatation, and coronary artery aneurysm, which may subsequently result in long-term sequelae like stenosis or obstruction and myocardial infarction. Accordingly, the decrease of the rate of coronary artery aneurysms among Kawasaki patients may be an indicator of the effectiveness of the treatment.

The ROCK2 inhibitors of the disclosure, including ROCK2 selective inhibitors, such as antisense RNA, siRNA or miRNA molecules or the compounds of Formula I to IV or Compound 1 as defined herein, can reduce pro-inflammatory cytokines, such as, for example, peripheral blood levels of IL-17 and IL-23. In addition, these ROCK2 inhibitors may rebalance the immune response in pediatric patients suffering from Kawasaki disease, PMIS, or PIMS-TS, after infection with COVID-19 or other coronaviruses.

### Methods of Treating Fibrosis Caused by Virus Infection

COVID-19 patients may develop scarring in their lungs either during the acute stage of the disease or after they recover from the illness. The scarring in the lung may be fibrosis (including post-COVID fibrosis) or fibrotic scarring. The underlying causes and the clinical course for scarring in the lung in COVID-19 patients may be the same as or different from those with interstitial lung disease and pulmonary fibrosis, such as idiopathic pulmonary fibrosis (IPF) or rheumatoid arthritis.

Fibrosis is the overgrowth, hardening, and/or scarring of various tissues and can be attributed to excess deposition of extracellular matrix components, such as, for example, collagen. Fibrosis may be the result of chronic inflammatory reactions induced by a variety of stimuli, including, for example, persistent infections, autoimmune reactions, allergic responses, chemical insults, radiation, and tissue injury.

A COVID-19 infection can lead to a variety of respiratory diseases ranging from atypical pneumonia to acute respiratory distress syndrome (ARDS). Many patients infected by the viruses may share the characteristics of patients suffering from IPF. For an IPF patient, lung function can inexorably decline, resulting in respiratory failure and death. A possible cause for the lung damage can be a cytokine release syndrome triggered by the viral antigen.

The ROCK2 signaling pathway controls cellular movement and shape. In addition, ROCK2 may regulate cytokine secretion in T cells, such as, for example, promoting pro-inflammatory cytokines such as IL-17 and IL-21, whereas secretion of anti-inflammatory cytokines IL-2 and IL-10 is negatively regulated by ROCK2 under Th17-skewing activation. Also, in disease, but not in steady state conditions, ROCK2 contributes to regulation of IFN-7 secretion in T cells from rheumatoid arthritis patients. Thus, ROCK2 signaling is a key pathway in modulation of T-cell mediated immune responses underscoring the therapeutic potential of targeted inhibition of ROCK2 in autoimmunity. Accordingly, ROCK2 inhibitors can down-regulate IL-21 and IL-17 secretion in human T cells via STAT3-dependent mechanism.

The ROCK2 inhibitors of the disclosure, including ROCK2 selective inhibitors, such as antisense RNA, siRNA or miRNA molecules or the compounds of Formula I to IV or Compound 1 as defined herein, can reduce peripheral blood levels of IL-17 and IL-23, two pro-inflammatory cytokines. In addition, these ROCK2 inhibitors can concurrently up-regulate the immunosuppressive cytokine IL-10 and increase the percentage of Foxp3+CD4 T cells in blood, which may diminish immuno-inflammatory responses. Thus, the ROCK2 inhibitors of the disclosure, including ROCK2 selective inhibitors, such as antisense RNA, siRNA or miRNA molecules or the compounds of Formula I to IV or Compound 1 as defined herein, can rebalance the immune response in patients suffering from fibrosis after infection with SARS-CoV-2 or other coronaviruses. Further, the ROCK2 inhibitors of the disclosure, including ROCK2 selective inhibitors, such as antisense RNA, siRNA or miRNA molecules or the compounds of Formula I to IV or Compound 1 as defined herein, can treat or prevent pulmonary fibrosis, including post-COVID fibrosis.

A number of pulmonary function parameters can be used to determine an effective amount of the ROCK2 inhibitor of the disclosure, i.e., an amount to reduce, stabilize or reverse a pathologic rate of decline in one or more pulmonary function parameters in a patient suffering from a viral infection, such as a coronavirus infection; or to monitor patient response to ROCK2 inhibitor therapy. These pulmonary function parameters may include vital capacity (VC), forced expiratory volume (FEV), forced vital capacity (FVC), and FVC %.

As used herein, the term “vital capacity” (VC) refers to the total volume of air that can be moved in and out of the lungs. VC is equal to the combined inspiratory reserve volume, tidal volume, and expiratory reserve volume.

As used herein, the term “forced expiratory volume” (FEV) refers to measuring how much air a subject can exhale during a forced breath. The amount of air exhaled may be measured during the first (FEV1), second (FEV2), and/or third seconds (FEV3) of the forced breath. For example, FEV1/FVC ratio refers to the ratio between forced expiratory volume in one second and forced vital capacity.

As used herein, the term “forced vital capacity” (FVC) refers to the vital capacity from a maximally forced expiratory effort, i.e., the total amount of air exhaled expelled by a subject during the FEV test.

As used herein, the term “FVC %” refers to the percent change in the FVC of a subject over a period of time. FVC % predicted is a subject's measured FVC expressed as the percentage of the predicted FVC for the subject. As used herein, all FVC % predicted values are absolute values and not relative value.

Many of these pulmonary function parameters are readily obtainable using a spirometer. Residual volume can be obtained through indirect methods such as radiographic planimetry, body plethysmography, closed circuit dilution (including the helium dilution technique), and nitrogen washout.

Lung capacity and associated pulmonary function parameters naturally decline due to aging. Numerous studies have been conducted among normal populations to determine the rate of decline of lung capacity and other pulmonary function parameters. See Crapo et al. (1981) Am. Rev. Respir. Dis. 123:659-664. For example, a 65 years-old Caucasian male is expected to have a decline of 0.03 liters in FVC at age 66.

In contrast to the natural decline due to aging, subjects with lung disease such as pulmonary fibrosis and fibrosis caused by COVID-19 have an abnormally steep rate of decline in lung capacity or in one or more pulmonary function parameters, i.e., a “pathologic rate of decline.” As used herein, a “pathologic rate of decline” is a rate of decline in lung capacity or in one or more pulmonary function parameters that is at least 5% greater than the decline due to normal aging. In some embodiments, a pathologic rate of decline is at least 5%, 10%, 15%, 20%, 25%, 30%, 40%, 50%, 60%, 70%, 80%, 90%, 100%, 125%, 150%, 200%, 300%, 400%, 500%, 600%, 700%, 800% or 1000% greater than the predicted rate of decline for a normal person of similarly matched race or ethnicity, gender, age, height, and weight. Rates of decline can be expressed as the change from baseline per 1 week, 2 weeks, 4 weeks, 8 weeks, 12 weeks, 24 weeks, 36 weeks, 48 weeks, or 12 months. In some embodiments, the pathologic rate of decline in lung capacity is the change in forced vital capacity (FVC) from baseline of at least about −0.05 liters, −0.10 liters, −0.15 liters, −0.20 liters or −0.25 liters per 12 months. In other embodiments, the pathological rate of decline is the change from baseline forced vital capacity percent (FVC %) predicted of at least about −2%, −3%, −4%, −5%, −6%, −7%, −8% or −10% per 12 months.

Administration of the ROCK2 inhibitors of the disclosure, including ROCK2 selective inhibitors, such as antisense RNA, siRNA or miRNA molecules or the compounds of Formula I to IV or Compound 1 as defined herein, or a pharmaceutically acceptable salt, ester, stereoisomer, polymorph, solvate, N-oxide, or isotopically labeled compound thereof, may result in an increase of FVC in a subject with fibrosis after the treatment vs. before the treatment. Treatment with an effective amount of the ROCK2 inhibitor may increase FVC by at least 0.5%, 1%, 1.5%, 2.0%, 2.5%, 3.0%, 4.0%, 5.0%, 6.0%, 7.0%, 8.0%, 9.0%, 10%, 15%, 20%, 30%, 40% or 50% compared to FVC before the treatment. In some embodiments, treatment with the ROCK2 inhibitor is for at least 1 week, 2 weeks, 3 weeks, 6 weeks, 9 weeks, 12 weeks, 15 weeks, 18 weeks, 21 weeks, 24 weeks, 27 weeks, 30 weeks, 33 weeks, 36 weeks or 48 weeks. In some embodiments, treatment is for 2 weeks or less, 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from starting treatment with the ROCK2 inhibitor.

### Kits

In one aspect, the present disclosure provides a kit or composition that includes a ROCK2 inhibitor of the disclosure, including ROCK2 selective inhibitors, such as antisense RNA, siRNA or miRNA molecules or the compounds of Formula I to IV or Compound 1 as defined herein, or a pharmaceutically acceptable salt, pharmaceutically acceptable ester, stereoisomer, mixture of stereoisomers or tautomer thereof. In some embodiments, individual kits are provided, including a compound of Formulas I to IV or Compound 1 as defined herein, or a pharmaceutically acceptable salt, pharmaceutically acceptable ester, stereoisomer, mixture of stereoisomers or tautomer thereof. In one aspect, the kit comprises a compound of Formulas I to IV or Compound 1, or a pharmaceutically acceptable salt thereof. Each of the individual kits described herein may comprise a label and/or instruction for use of the compound in the treatment of a disease or condition in a subject (e.g., human) in need thereof. In some embodiments, the disease or condition is a coronavirus infection. In other embodiments, each separate kit may also contain instructions for use of additional medical agents in combination with the ROCK2 inhibitor of the disclosure, including ROCK2 selective inhibitors, such as antisense RNA, siRNA or miRNA molecules or the compounds of Formula I to IV or Compound 1 as defined herein, in the treatment of a disease or condition in a subject (e.g., human) in need thereof. In some embodiments, the kit comprises belumosudil. In some embodiments, the kit comprises individual dose units of a ROCK2 inhibitor, including a ROCK2 selective inhibitor, described herein. Examples of individual dosage units may include pills, tablets, capsules, prefilled syringes or syringe cartridges, IV bags, etc., each comprising a therapeutically effective amount of the ROCK2 inhibitor in question, or a pharmaceutically acceptable salt, racemate, enantiomer, diastereomer, tautomer, polymorph, pseudopolymorph, amorphous form, hydrate or solvate thereof. In some embodiments, the kit may contain a single dosage unit and in others multiple dosage units are present, such as the number of dosage units required for a specified regimen or period.

Also provided are articles of manufacture that include a ROCK2 inhibitor of the disclosure, including ROCK2 selective inhibitors, such as antisense RNA, siRNA or miRNA molecules or the compounds of Formula I to IV or Compound 1 as defined herein, or a pharmaceutically acceptable salt, pharmaceutically acceptable ester, stereoisomer, mixture of stereoisomers or tautomer thereof; and a container. In one aspect, the article of manufacture comprises a compound of Formulas I to IV or Compound 1, or a pharmaceutically acceptable salt thereof, and a container. In some embodiments, the article of manufacture comprises belumosudil. In some embodiments, the container of the article of manufacture is a vial, jar, ampoule, preloaded syringe, blister package, tin, can, bottle, box, or an intravenous bag.

### Exemplary Embodiments

Particular embodiments of the disclosure are set forth in the following numbered paragraphs:
1. A method for treating a viral infection in a subject in need thereof by administering to the subject a therapeutically effective amount of a ROCK2 inhibitor.
2. A method for preventing a viral infection in a subject at risk for the viral infection by administering to the subject a prophylactically effective amount of a ROCK2 inhibitor.
3. A method of treating Kawasaki disease, PMIS, or PIMS-TS associated with a viral infection in a subject in need thereof, comprising administering to the patient a therapeutically effective amount of a ROCK2 inhibitor.
4. A method for the treatment or prevention of sequelae resulting from a viral infection in a subject in need thereof, comprising administering to the patient a therapeutically effective amount of a ROCK2 inhibitor.
5. The method of any one of paragraphs 1-4, wherein the ROCK2 inhibitor is a ROCK2 selective inhibitor.
6. The method of any one of paragraphs 1-5, wherein the ROCK2 inhibitor is a ribonucleic acid (RNA).
7. The method of paragraph 6, wherein the ROCK2 inhibitor is an antisense RNA, a small interfering RNA or a microRNA.
8. The method of any one of paragraphs 1-5, wherein the ROCK2 inhibitor is a compound having the structure of Formula I:

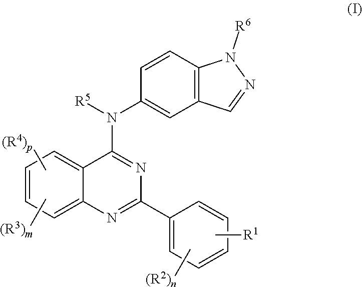

or a pharmaceutically acceptable salt, ester, stereoisomer, polymorph, solvate, N-oxide, or isotopically labeled compound thereof, wherein:
R¹ is selected from the group consisting of O—(CH₂)ᵣCO₂R¹², —O—(CH₂)_{y}, —C(═O)NR¹³R¹⁴, —O—(CH₂)_{y}, -heteroaryl, —O—(CH₂)_{y}, -cycloalkyl, —O—C(═O)—(CH₂)_{y}, —NR¹³R¹⁴, —O—(CH₂)_{z}—NR¹³R¹⁴, —NH—C(═O)—(CH₂)_{y}, —NR¹³R¹⁴, —NH—C(═O)—X—R¹⁵, and —NH—(CH₂)_{y}, —NR¹³R¹⁴;
R¹² is selected from the group consisting of C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, —(C₁-C₆ alkyl)-O—(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl;
R¹³ and R¹⁴ are independently selected from the group consisting of H, C₁-C₈alkyl, C₂-C₈ alkenyl, C₂-C₈ alkynyl, —(C₁-C₆ alkyl)—O—(C₁-C₆ alkyl), —(C₁-C₆ alkyl)-NR¹⁶R¹⁷, —(C₁-C₆ alkyl)-C(═O)NR¹⁶R¹⁷ aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
X is absent or selected from a —O, NH, and C₁-C₆ alkyl;
R¹⁵ is selected from the group consisting of heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁵ is selected from —(C₁-C₆ alkyl)—O—(C₁-C₆ alkyl), —(C₁-C₆ alkyl)-NR¹⁶R¹⁷, —CO₂R¹⁸, —O—(CH₂)ₓ—CO₂R¹⁸, and —C(═O)NR¹⁶R¹⁷;
R¹⁶ and R¹⁷ are independently selected from the group consisting of H, C₁-C₈ alkyl, C₂-C₈ alkenyl, C₂-C₈ alkynyl, —(C₁-C₆ alkyl)—O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R¹⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR¹⁶R¹⁷, —(C₁-C₆ alkyl)—O—(C₁-C₆ alkyl)—O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
x is selected from 0 to 6;
y is selected from 0 to 6;
z is selected from 2 to 6;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
R⁴ is selected from H, —(CH₂)ₐ—NR⁴³R⁴⁴, —Y—R⁴², —O—(CH₂)ₐ—CO₂R⁴², —O—(CH₂)ₐ—C(═O)NR⁴³R⁴⁴, —O—(CH₂)ₐ-heteroaryl, —O—(CH₂)ₐ-cycloalkyl, —O—C(═O)—(CH₂)ₐ—NR⁴³R⁴⁴, —O—(CH₂)_{c}—N⁴³R⁴⁴, —NH—C(═O)—Y—R⁴, —NH—C(═O)—(CH₂)ₐ—NR⁴³R⁴⁴;
R⁴² is selected from the group consisting of C₁-C₆ alkyl, —(C₁-C₆ alkyl)—O—(C₁-C₆ alkyl), —(C₁-C₆ alkyl)-NR⁴⁶R⁴⁷, —(C₁-C₆ alkyl)-C(═O)N⁴⁶R⁴⁷, —(C₁-C₆ alkyl)-O—(C₁—C₆ alkyl)-O—(C₁-C₆ alkyl), each of which may be optionally substituted at one or more carbon atoms by from 1 to 3 substituents independently selected from halo, C₁-C₆ alkoxy, hydroxy, amino, cyano and C₁-C₃ perfluoro alkyl;
R⁴³ and R⁴⁴ are independently selected from the group consisting of H, C₁-C₈ alkyl, C₂-C₈ alkenyl, C₂-C₈ alkynyl, —(C₁-C₆ alkyl)—O—(C₁-C₆ alkyl), —(C₁-C₆ alkyl)-NR⁴⁶R⁴⁷, —(C₁-C₆ alkyl)-C(═O)NR⁴⁶R⁴⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁴³ and R⁴⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
Y is absent or selected from O, NH, and C₁-C₆ alkyl;
R⁴⁵ is selected from the group consisting of H, aryl, —(C₁-C₆ alkyl)-O—(C₁-C₆ alkyl), —(C₁-C₆ alkyl)-NR⁴⁶R⁴⁷, —CO₂R⁴⁸, —O—(CH₂)₆—CO₂R⁴⁸, and —C(═O)NR⁴⁶R⁴⁷;
R⁴⁶ and R⁴⁷ independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁴⁶ and R⁴⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁴¹ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR⁴⁶R⁴⁷, —(C₁-C₆ alkyl)—O—(C₁—C₆ alkyl)—O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
a is selected from 0 to 6;
b is selected from 0 to 6;
c is selected from 2 to 6;
R⁵ is selected from the group consisting of H, C₁-C₆ alkyl, —(CH₂)_{d}—C(═O)—NR⁵³R⁵⁴, —C(═O)—(CH₂)_{d}—NR⁵³R⁵⁴, and —C(═O)—X—R⁵⁵;
R⁵³ and R⁵⁴ are independently selected from the group consisting of H, C₁-C₈ alkyl, C₂-C₈ alkenyl, C₂-C₈ alkynyl, —(C₁-C₆ alkyl)—O—(C₁-C₆ alkyl), —(C₁-C₆ alkyl)-NR⁵⁶R⁵⁷, —(C₁-C₆ alkyl)-C(═O)NR⁵⁶R⁵⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁵³ and R⁵⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁵⁵ is selected from the group consisting of H, aryl, —(C₁-C₆ alkyl)—O—(C₁-C₆ alkyl), —(C₁-C₆ alkyl)-NR⁵⁶R⁵⁷, —CO₂R⁵⁸, —O—(CH₂)_{c}—CO₂R⁵⁸, and —C(═O)NR⁵⁶R⁵⁷;
R⁵⁶ and R⁵⁷ independently selected from the group consisting of H, C₁-C₈ alkyl, C₂-C₈ alkenyl, C₂-C₈ alkynyl, —(C₁-C₆ alkyl)—O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁵⁶ and R⁵⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁵⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR⁵⁶R⁵⁷, —(C₁-C₆ alkyl)—O—(C₁-C₆ alkyl)—O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
d is selected from 0 to 6;
e is selected from 0 to 6;
R⁶ is selected from the group consisting of H, C₁-C₆ alkyl, —(CH₂)ᵣ—C(═O)—NR⁶³R⁶⁴, C(═O) (CH₂)ᵣ—NR⁶³R⁶⁴, and —C(═O)—X—R⁶⁵;
R⁶³ and R⁶⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁶⁶R⁶⁷, —(C₁-C₆ alkyl)-C(═O)NR⁶⁶R⁶⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁶³ and R⁶⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁶⁵ is selected from the group consisting of H, aryl, —(C₁-C₆ alkyl)-O—(C₁-C₆ alkyl), —(C₁-C₆ alkyl)-NR⁶⁶R⁶⁷, —CO₂R, —O—(CH₂), —CO₂R⁶⁸, and —C(═O)NR⁶⁶R⁶⁷.
R⁶⁶ and R⁶⁷ independently selected from the group consisting of H, C₁-C₅ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁶⁶ and R⁶⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁶⁸ is selected from the group consisting of H, aryl, aralkyl, heteroaryl, C₁-C₆ alkyl, —(C₁-C₆ alkyl-O—(C₁-C₆ alkyl), —(C₁-C₆ alkyl)-NR⁶⁶R⁶⁷, —(C₁-C₆ alkyl)—O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
r is selected from 0 to 6;
s is selected from 0 to 6;
n is selected from 0 to 4;
m is selected from 0 to 3; and
p is selected from 0 and 1.
9. The method of paragraph 8, wherein the ROCK2 inhibitor is a compound having the structure of Formula II:

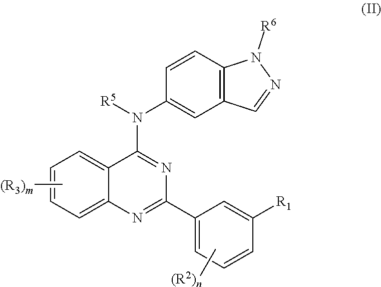

or a pharmaceutically acceptable salt, ester, stereoisomer, polymorph, solvate, N-oxide, or isotopically labeled compound thereof, wherein: R¹, R², R³, R⁵, R⁶, m and n are as defined in paragraph 8.
10. The method of paragraph 9, wherein the ROCK2 inhibitor is a compound having the structure of Formula III:

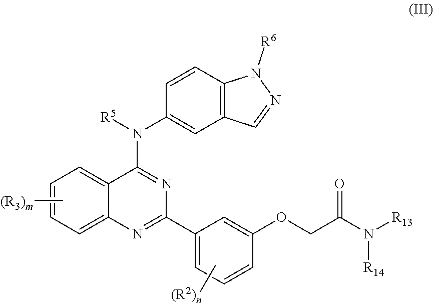

or a pharmaceutically acceptable salt, ester, stereoisomer, polymorph, solvate, N-oxide, or isotopically labeled compound thereof, wherein:
R¹³ and R¹⁴ are independently selected from the group consisting of H, C₁-C₈alkyl, C₂-C₈ alkenyl, C₂-C₈ alkynyl, —(C₁-C₆ alkyl)—O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
wherein each of R², R³, R⁵, R⁶, m and n are as defined in paragraph 8.
11. The method of paragraph 10, wherein the ROCK2 inhibitor is a compound having the structure of Formula IV:

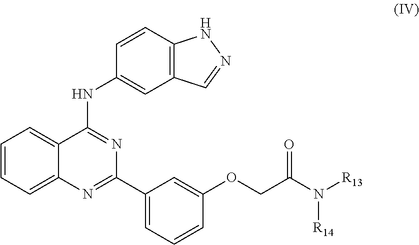

or a pharmaceutically acceptable salt, ester, stereoisomer, polymorph, solvate, N-oxide, or isotopically labeled compound thereof, wherein: R¹³ and R¹⁴ are as defined in paragraph 10.
12. The method of paragraph 11, wherein the ROCK2 inhibitor is the compound (2-(3-(4-((1H-indazol-5-yl)amino)quinazolin-2-yl)phenoxy)-N-isopropylacetamide), having the chemical structure of Compound 1:

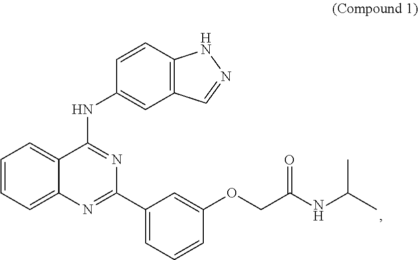

or a pharmaceutically acceptable salt, ester, stereoisomer, polymorph, solvate, N-oxide, or isotopically labeled compound thereof.
13. The method according to any one of paragraphs 1 to 12, wherein the viral infection is caused by a coronavirus.
14. The method according to any one of paragraphs 1 to 13, wherein the viral infection is caused by SARS-CoV-1, SARS-CoV-2 or MERS-CoV.
15. The method according to any one of paragraphs 1 to 14, wherein the viral infection is caused by SARS-CoV-2.
16. The method according to paragraph 15, wherein the viral infection is caused by a Delta or Omicron variant of SARS-CoV-2.
17. The method according to any one of paragraphs 4 to 16, wherein the sequelae resulting from the viral infection include one or more of the group consisting of fatigue, dyspnea, cough, arthralgia, myalgia, headache, chest pain, fever, palpitations, myocardial inflammation, ventricular dysfunction, stroke, pulmonary function abnormalities, pulmonary fibrosis, renal dysfunction rash, alopecia, olfactory and/or gustatory dysfunction, sleep dysregulation, cognitive impairment altered, memory impairment, depression, anxiety, changes in mood, and combinations thereof.
18. The method according to any one of paragraphs 4 to 17, wherein the sequelae is pulmonary fibrosis.
19. The method according to any one of paragraphs 1 to 18, wherein the method further comprises administering to the subject a therapeutically effective amount of at least one other therapeutic agent.
20. The method according to paragraph 19, wherein the at least one other therapeutic agent is selected from the group consisting of an antiviral agent, a corticosteroid, an anti-inflammatory signal transduction modulator, a β2-adrenoreceptor agonist bronchodilator, an anticholinergic, a mucolytic agent, hypertonic saline, and combinations thereof.
21. The method according to paragraph 19, wherein the at least one other therapeutic agent comprises remdesivir.

### EXAMPLES

The following illustrative examples are representative of embodiments of compositions and methods described herein and are not meant to be limiting in any way.

### Example 1. In Vitro SARS-CoV-2 Prevention Model

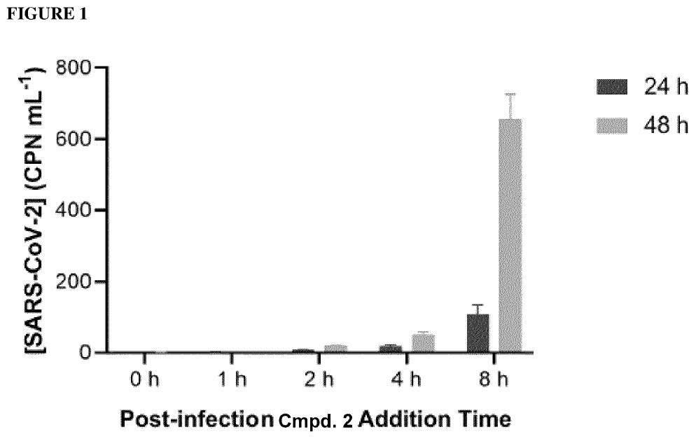

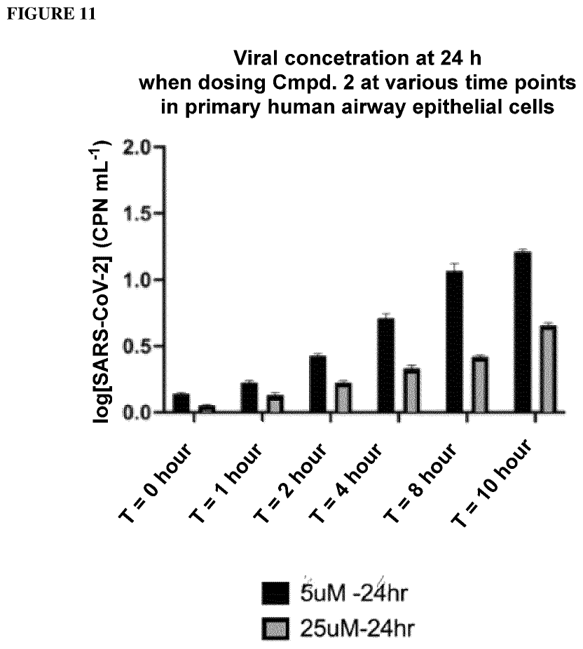

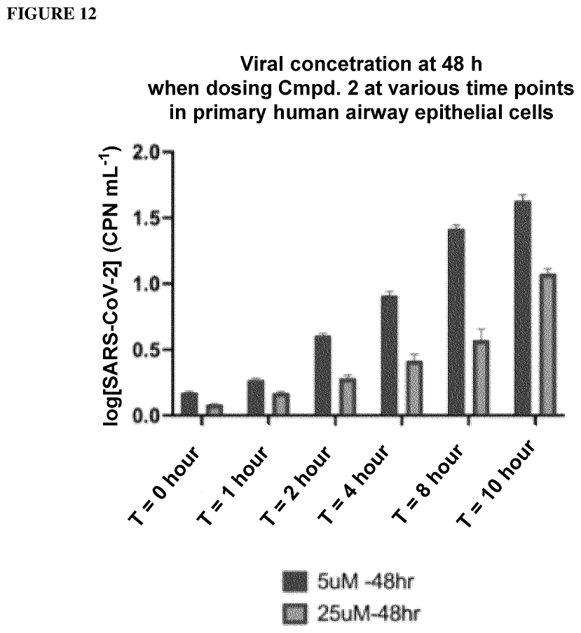

A 50 mM stock solution of Compound 2 (in DMSO) was diluted using PBS to concentrations of 100, 75, 50, 25, 10, 5, 1, 0.3, 0.1, and 0.03 μM. About 30,000 immortalized human airway epithelial cells (Calu-3) (see, FIG. 1-10) or primary human airway epithelial cells (Normal Human Small Airway Epithelial Cells; Sigma) (see, FIGS. 11-12) were grown to about 100,000 cells per well in a 96-well format. The cells were challenged with 10² or 10³ PFU SARS-CoV-2 (standard CDC SARS-CoV-2 strain) in triplicate and incubated for 6 h at 37° C., followed by washing. The cells were then treated with Compound 2 or negative control by adding 10 μL of a solution of Compound 2 (0.03-100 μM) or negative control solution (DMSO in PBS) in triplicate and incubating for 2 h at 37° C. A positive control consisted of remdesivir (RDV; 10 μM). An additional control (3 wells) was treated with Compound 2 at the highest concentration, but not challenged with virus. The cells were maintained until the second endpoint readout at 48 h (the first occurring at 24 h post-infection). In some instances, the cellular media was replaced 24 h after infection with untreated culture medium (“unmediated medium”) or culture medium containing the positive control (RDV) or Compound 2 (“mediated medium”), see, FIG. 10.

Viral infection/load was analyzed by qPCR (qRT-PCR). RNA from cells was extracted with TRIzol reagent (Thermo Fisher) according to manufacturer's instructions. Viral or host RNA levels in the supernatant were determined using the TaqPath™ 1-Step RT-qPCR Master Mix (Thermo Fisher) on a CFX Connect Real-Time System (Bio-Rad) instrument using the following primers: Fwd: 5′-GACCCCAAAATCAGCGAAAT-3′; Rev. 5′-TCTGGTTACTGCCAGTTGAATCTG-3′.

The toxicity of Compound 2 was measured at concentrations of 0.1, 0.3, 1, 10, 25, 50, 75, and 100 μM in Calu-3 cells by LDH (lactase dehydrogenase) assay (Clontech) according to the manufacturer's instructions. A positive control consisted of saponin (0.5%). A negative control consisted of DMSO (0.2%).

Datasets were analyzed and plotted in GraphPad Prism (version 9.0.2). Analytic simulations of dose-response curves using the median-effect principle and mass-action Law were carried out using CompuSyn (https://www.combosyn.com/). Results of the in vitro studies are provided in FIGS. 1-12 and demonstrate the ability of Compound 2 to substantially reduce viral titers in immortalized human airway epithelial cells infected with SARS-CoV-2, as well as its low cellular toxicity.

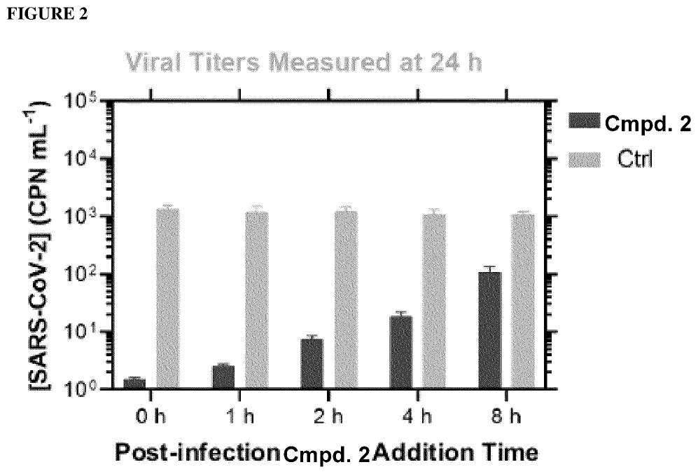

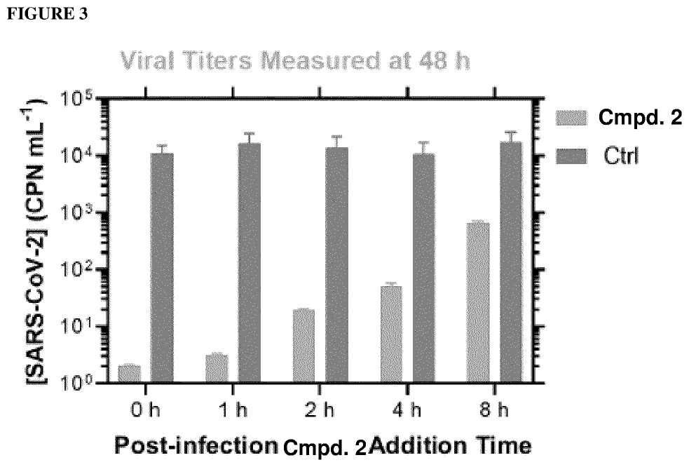

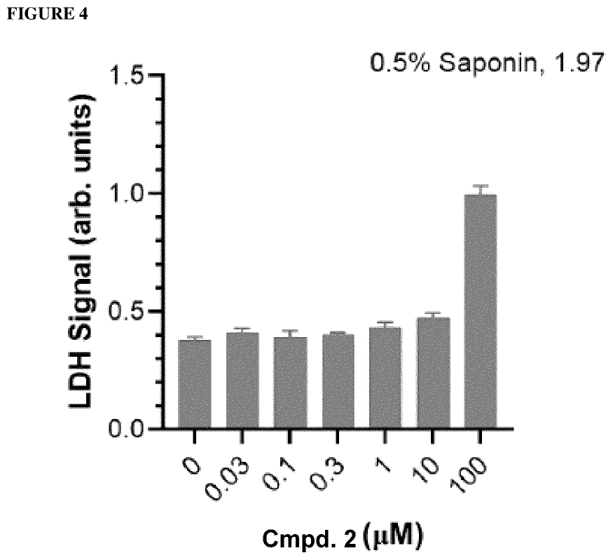

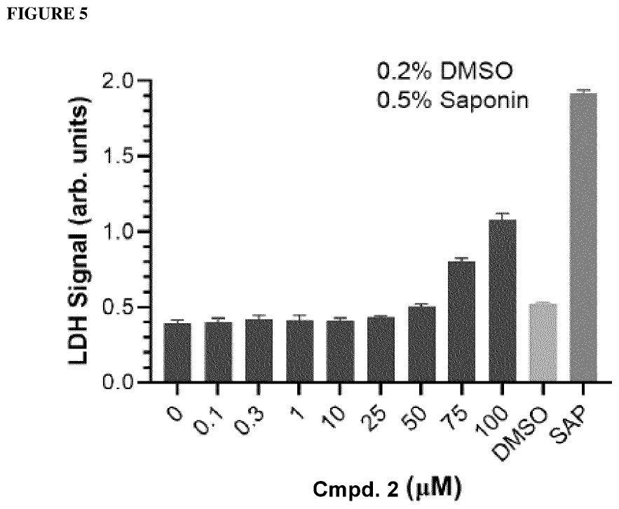

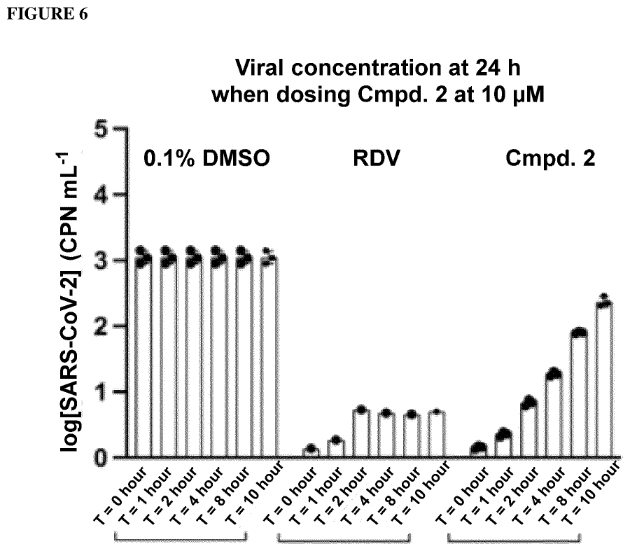

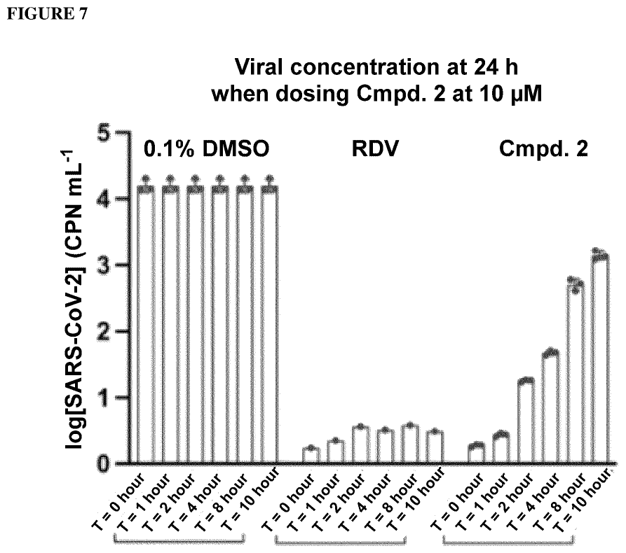

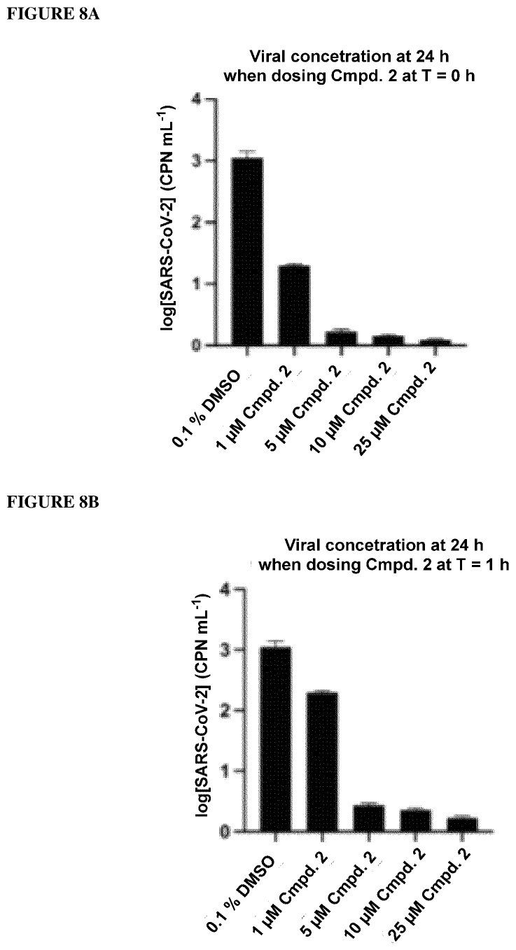

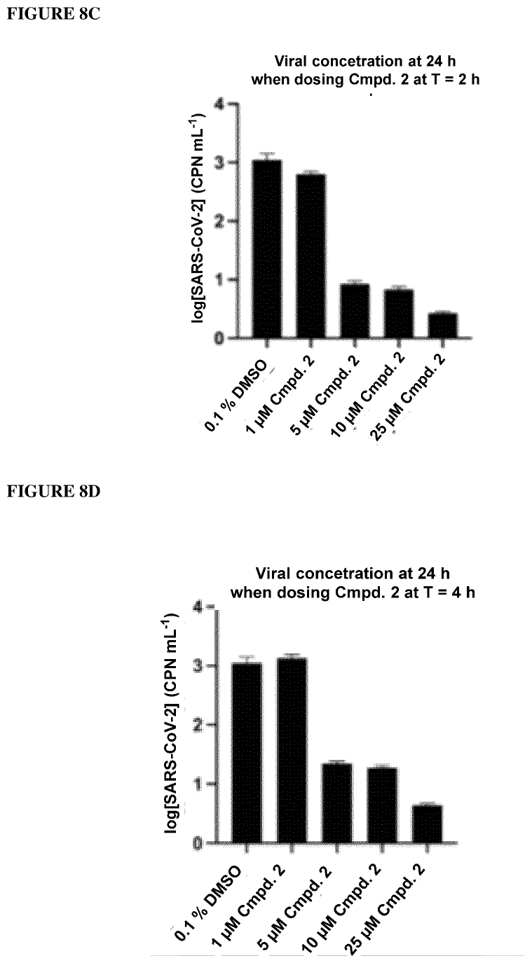

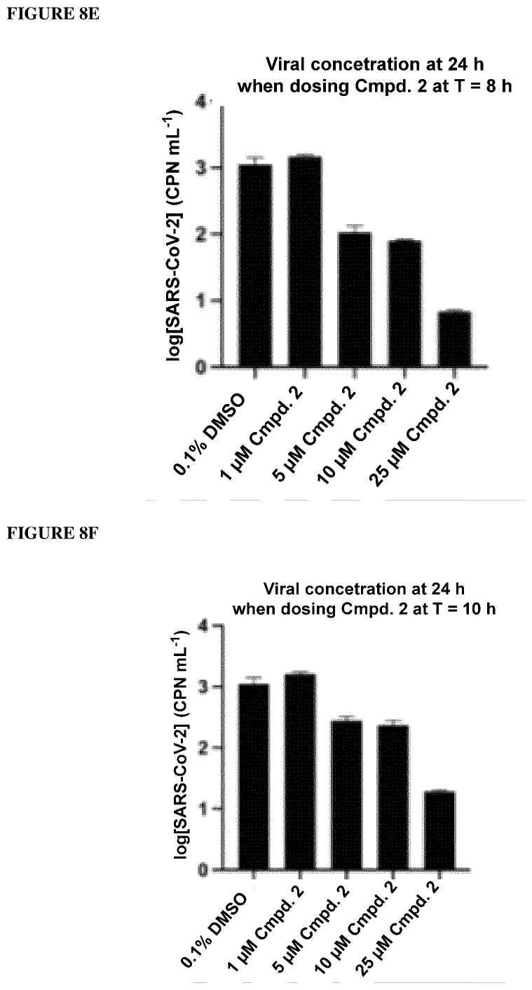

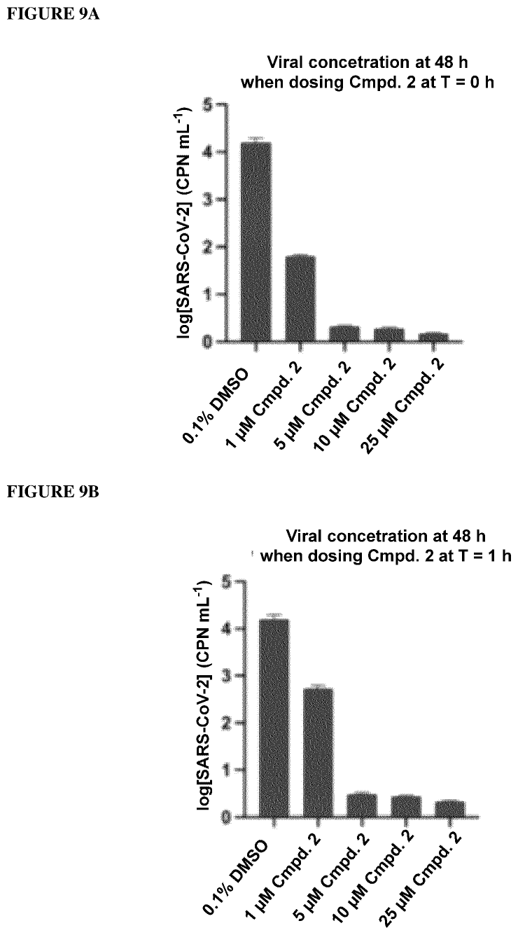

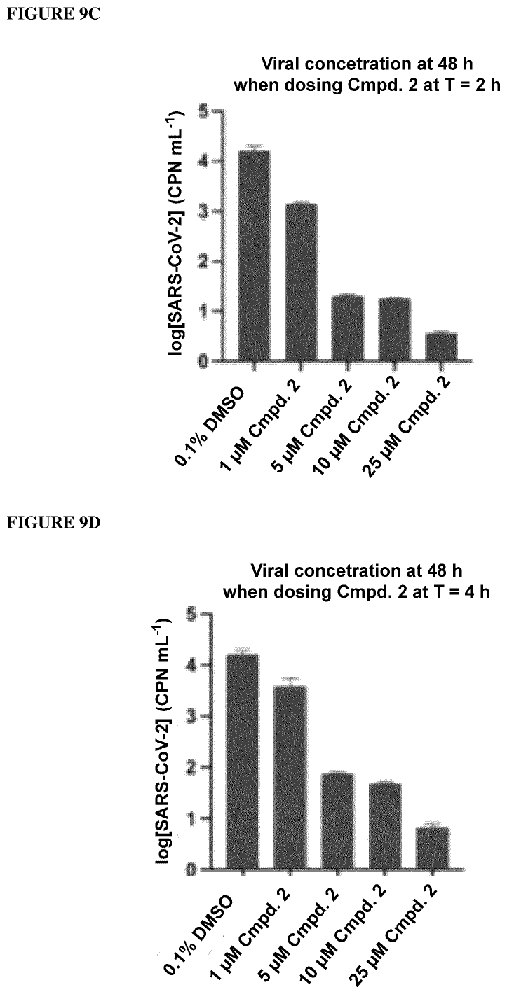

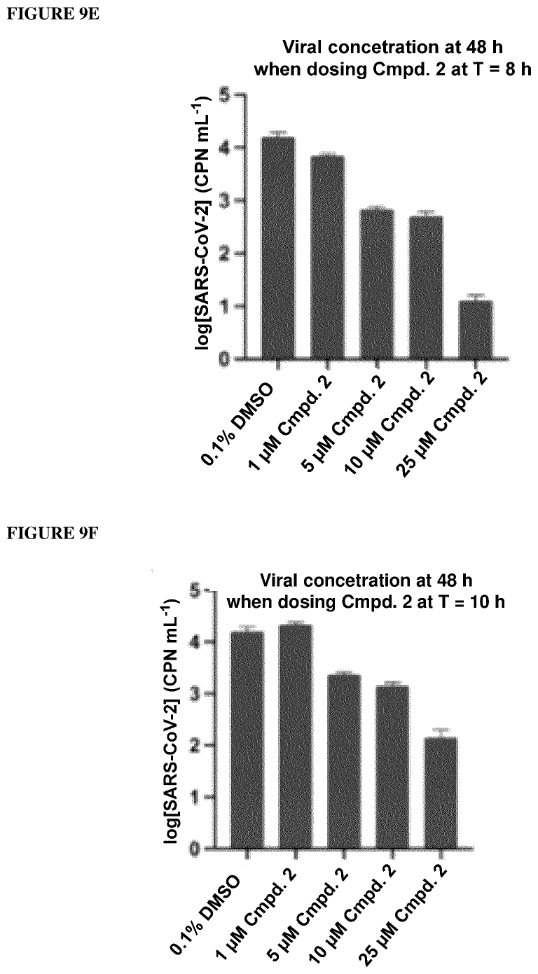

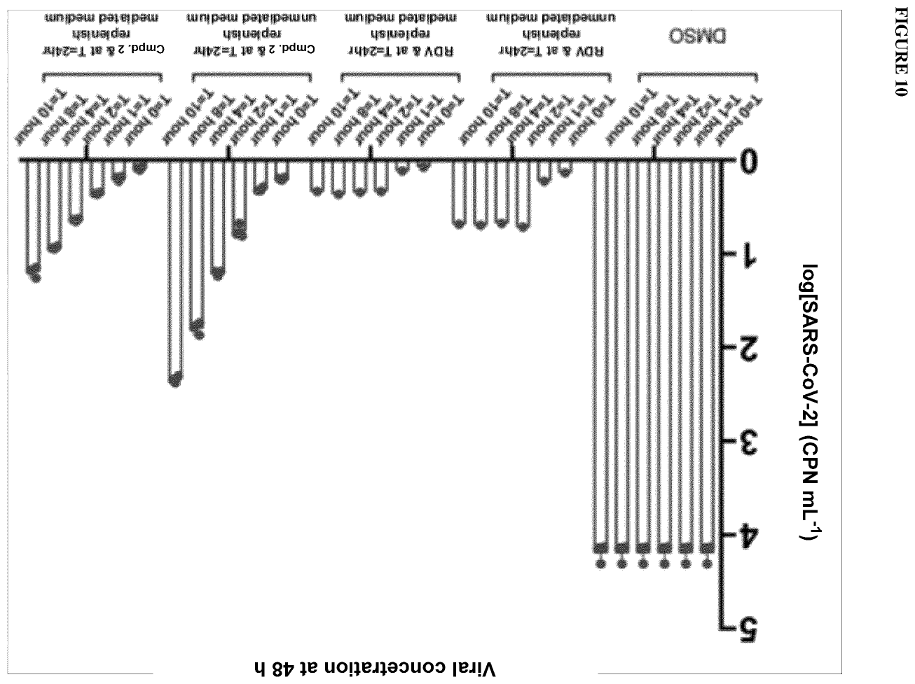

For example, FIGS. 1-3 demonstrate the in vitro efficacy of Compound 2 (10 μM) in inhibiting viral replication after 24 and 48 hours (FIG. 1), 24 hours (FIG. 2), and 48 hours (FIG. 3) as compared to control and that the efficacy correlated with administration time. FIGS. 4 and 5 demonstrate the low cellular toxicity of Compound 2 at various concentrations (0.03, 0.1, 0.3, 1, 10, 25, 50, 75, and 100 μM) as measured by LDH. FIGS. 6 and 7 demonstrate the in vitro efficacy of Compound 2 (10 μM) in inhibiting viral replication after 24 hours (FIG. 6) and 48 hours (FIG. 7) as compared to a negative control (0.1% DMSO) and a positive control (RDV). FIGS. 8A-8F demonstrate the ability of various doses (1 μM, 5 μM, 10 μM, and 25 μM) of Compound 2 to inhibit viral replication after 24 hours correlates with the administration time after infection—0 hours (FIG. 8A); 1 hour (FIG. 8B); 2 hours (FIG. 8C); 4 hours (FIG. 8D); 8 hours (FIG. 8E) and 10 hours (FIG. 8F). FIGS. 9A-9F demonstrate the ability of various doses (1 μM, 5 μM, 10 μM, and 25 μM) of Compound 2 to inhibit viral replication after 48 hours correlates with the administration time after infection—0 hours (FIG. 9A); 1 hour (FIG. 9B); 2 hours (FIG. 9C); 4 hours (FIG. 9D); 8 hours (FIG. 9E) and 10 hours (FIG. 9F). Dose responses were observed at all time points. FIG. 10 demonstrates that repeat dosing of Compound 2 further decreased viral concentration. FIGS. 11 and 12 demonstrate Compound 2's efficacy at 5 μM and 25 μM concentrations in treating a SARS-CoV-2 infection of primary human airway cells after 24 hours (FIG. 11) and 48 hours (FIG. 12) correlates with the administration time following infection.

### Example 2. Compound 2 Reduces the Spread of SARS-CoV-2 in the Lung In Vivo

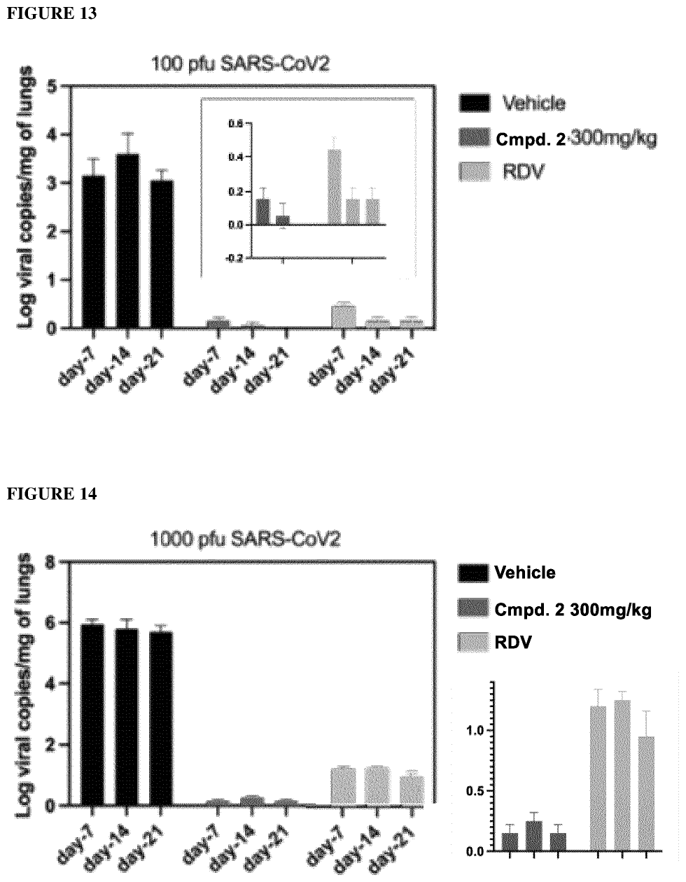

Human ACE2 transgenic mice from Jackson Laboratories (n=5 or n=8 per group) were challenged at t=0 with SARS-CoV-2 at an initial viral load of 100 or 1,000 pfu of 2019-nCoV/USA-WA1/2020 strain (or, alternatively, Delta or Omicron variants). The WA1/2020 strain was isolated from an oropharyngeal swab from a patient with a respiratory illness who developed the clinical disease (COVID-19) in January 2020 in Washington, US (BEI Resources, NR-52281). A viral load of 10,000 pfu is considered to be significantly higher than the corresponding viral dose a human subject would encounter in nature. The infected mice were dosed daily via oral gavage with: (1) 0.5% sodium carboxymethyl cellulose (CMC-Na) (negative control), (2) 100 and/or 300 mg/kg Compound 2 in 0.5% CMC-Na (test group), or (3) Remdesivir (RDV). RDV, an anti-viral reagent that acts as a nucleoside analog and inhibits the RNA-dependent RNA polymerase (RdRp) of coronaviruses included SARS-CoV-2, was used a positive control. RDV was solubilized at 2.5 mg/mL in vehicle containing 12% sulfobutylether-β-cyclodextrin sodium salt in water (with HCl/NaOH) at pH 5.0 (25 mg/kg subcutaneously) and continued every day until the end of the study. The animals were monitored daily. Two animals were sacrificed at the indicated times (X-axis of FIGS. 13, 14, 15A and 15B). The viral load in the lung was determined by qPCR detecting viral RNA in lung lysates. Hematoxylin and eosin (H & E) staining was used to assess lung fibrosis (FIG. 16).

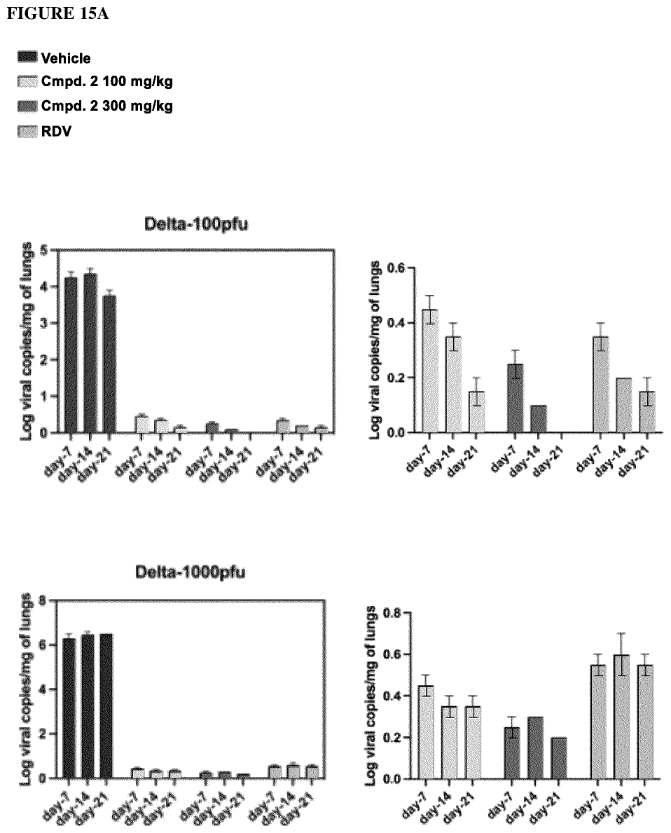

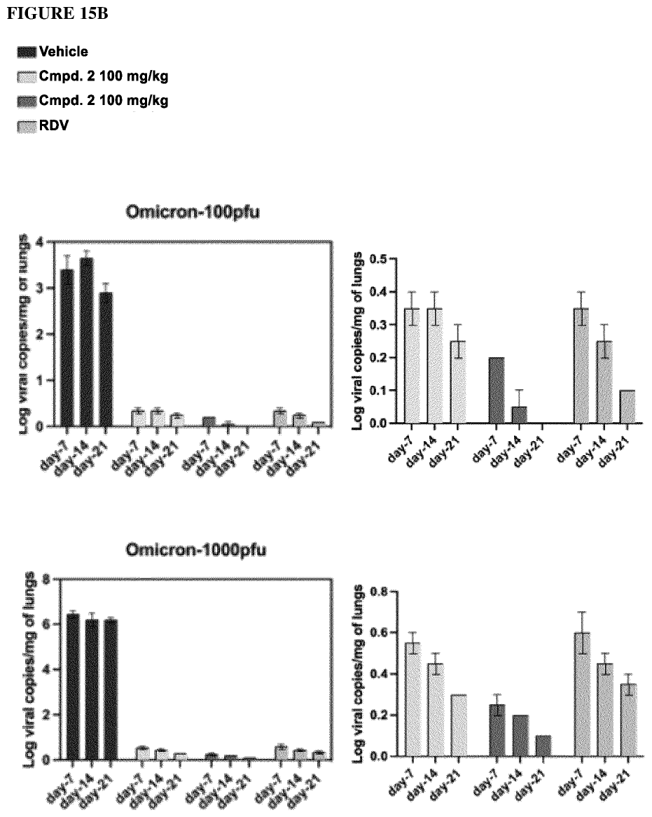

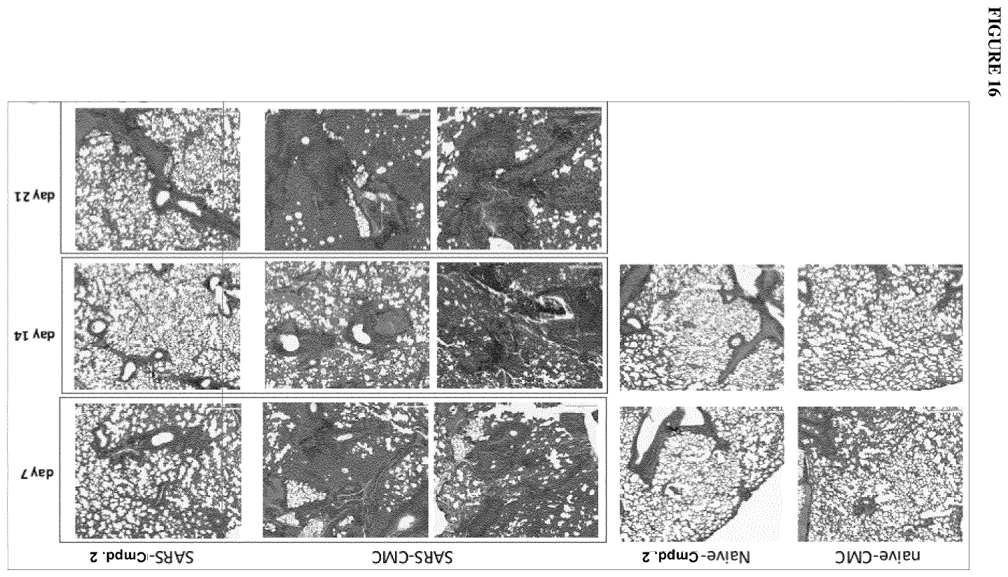

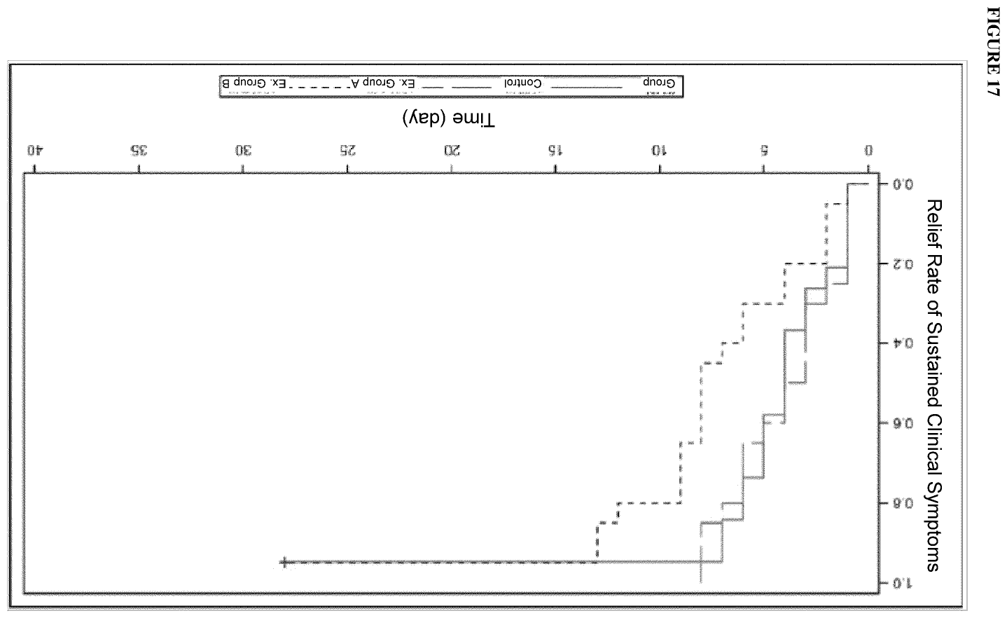

As shown in FIGS. 13 and 14, treatment with Compound 2 led to a significantly reduced viral load as compared to both the negative and the positive control. These data demonstrate that Compound 2 prevented SARS-CoV-2 cell entry, which requires the actin cytoskeleton And, as shown in FIGS. 15A and 15B, that effect was observed against both the Delta (FIG. 15A) and Omicron (FIG. 15B) variants of SARS-CoV-2. Moreover, as demonstrated by FIG. 16, treatment with Compound 2 had the additional benefit of reducing pulmonary fibrosis, which may help prevent long COVID.

### Example 3. Clinical Treatment of COVID-19 Using ROCK2 Inhibitor

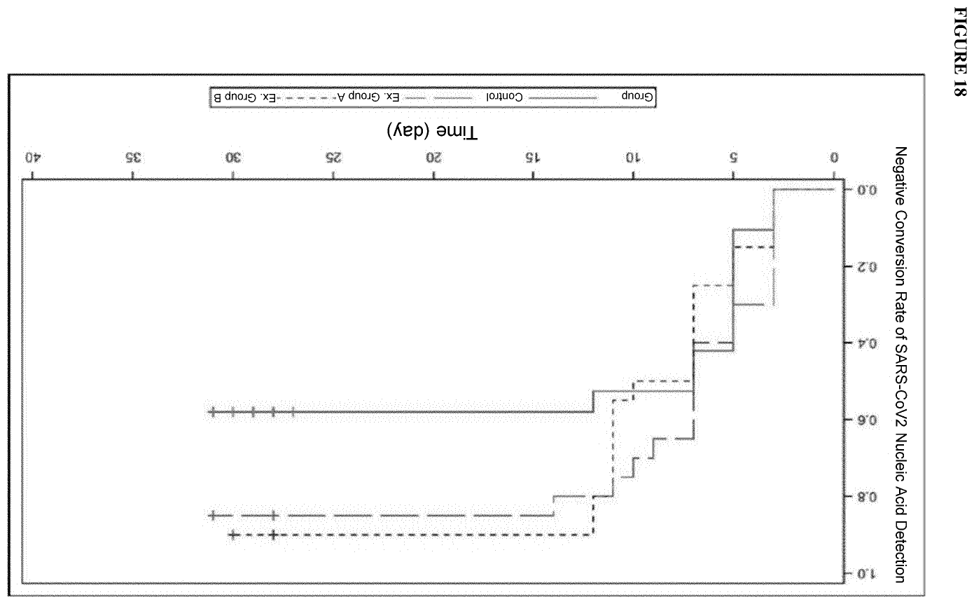

A 7-day clinical trial evaluating the efficacy of Compound 2 in treating patients with mild to moderate COVID-19 was conducted. Participants were selected for the trial after receiving a positive test result for SARS-CoV-2 virus and were administered their first dose of a placebo or Compound 2 (200 mg or 400 mg) less than 72 h after receiving their first positive test. In total, 90 patients participated in the study and were divided into 3 equally-sized study groups (i.e., 30 patients/group): Control, Experimental Group A, and Experimental Group B. Patients in the Control group received the standard of care for mild to moderate COVID-19 and a placebo once daily for 7 days. Patients in Experimental Group A received the standard of care for mild to moderate COVID-19 and Compound 2 (200 mg) once daily for 7 days. Patients in Experimental Group B received the standard of care for mild to moderate COVID-19 and Compound 2 (400 mg) once daily for 7 days. Patient viral load was measured daily to assess time to virus clearance, and clinical symptoms were observed and scored according to determine the percentage of participants with clinical symptom resolution. The results of the clinical trials are provided in FIGS. 17-20, which demonstrate the clinical efficacy of Compound 2 in treating mild to moderate COVID-19.

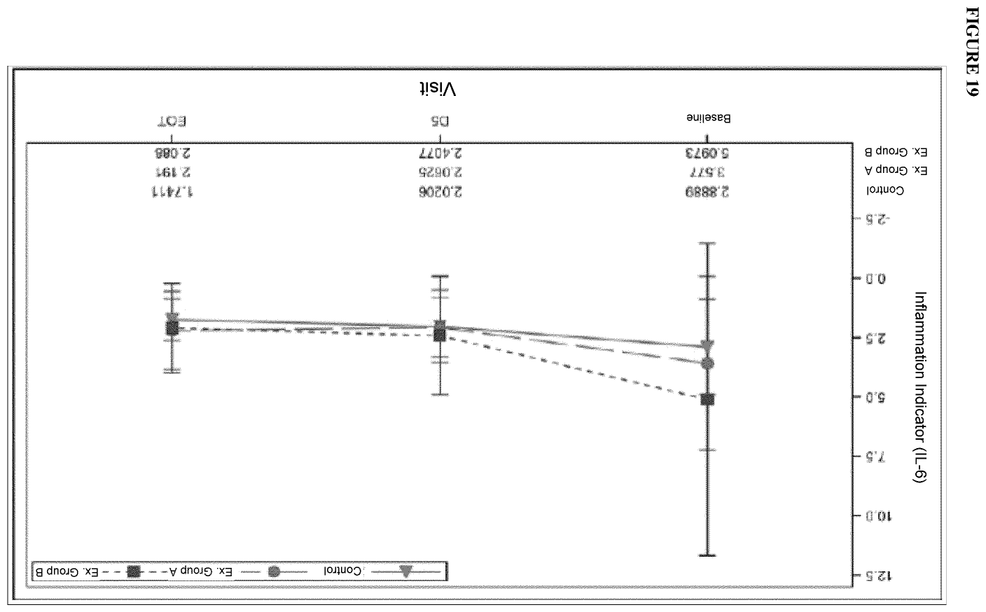

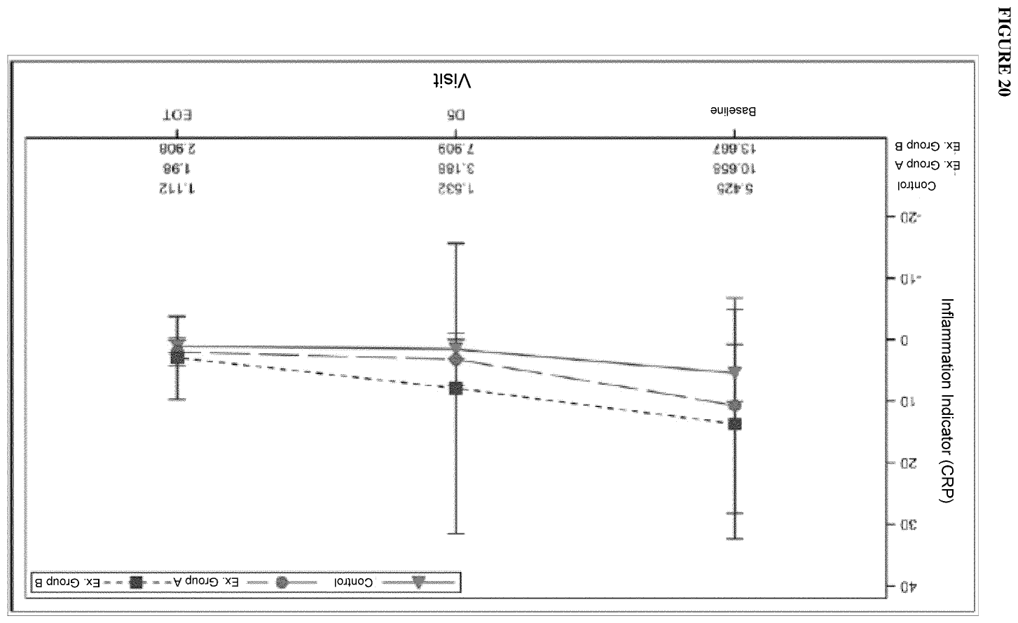

Specifically, FIG. 17 demonstrates that treatment with 400 mg of Compound 2 once daily (short dashes—Ex. Group B) led to a substantial improvement in the relief rate of sustained clinical symptoms relative to treatment regimens that included just the standard of care treatment (solid line (Control)) or the standard of care treatment in combination with daily administration of Compound 2 at a lower dose (long dashes—Ex. Group A). FIG. 18 shows that treatment with 200 mg (long dashes—Ex. Group A) or 400 mg (short dashes—Ex. Group B) of Compound 2 once daily led to lower viral titers in patients, as measured by negative conversion rates of SARS-CoV-2 nucleic acid detection, relative to Control (solid line). FIGS. 19 and 20 demonstrate that treatment with Compound 2 (circle, long dashes (Ex. Group A—200 mg/day)) (square, short dashes (Ex. Group B—400 mg/day)) reduced levels of inflammation in patients, as measured by quantifying levels of interleukin 6 (TL-6) and C-reactive protein (CRP), respectively.

### Example 4. Treatment of COVID-19 Using ROCK2 Inhibitor

A study is conducted in men and women suspected of having or having SARS-CoV-2 infections. The study employs (2-(3-(4-((1H-indazol-5-yl)amino)quinazolin-2-yl)phenoxy)-N-isopropylacetamide) (Compound 1), which is orally administered 100-400 mg daily for 2-3 weeks of the treatment. Various amounts of Compound 1 are administered at the designated intervals.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3 mL of either viral transport medium (VTM), Amies transport medium, or sterile saline, unless using a point-of-care test for coronaviruses. The sample is tested using a CDC authorized testing method. Different dosages of Compound 1 administered 100 mg/day, 200 mg/day, 300 mg/day, or 400 mg/day are also tested in patients following the same protocols and record the dose-response relationship for the extent of virus inhibition.

### Example 5. Treatment of COVID-19 Using ROCK2 Inhibitor and Remdesivir

A study is conducted on men and women suspected of having, or having, SARS-CoV-2 infections. The study employs Compound 1, which is orally administered 100-400 mg daily for 2-3 weeks of the treatment. In parallel, remdesivir is administered via intravenous infusion in a total volume of up to 250 mL 0.9% saline over 30 to 120 minutes for adult patients according to the following schedule for adult and pediatric patients with body weight of at least 40 kg: on day 1, loading dose of 200 mg; on days 2-10, once-daily dose of about 100 mg.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3 mL of either viral transport medium (VTM), Amies transport medium, or sterile saline, unless using a point-of-care test for coronaviruses. The sample is tested using a CDC authorized testing method. Different dosages of Compound 1 administered 100 mg/day, 200 mg/day, 300 mg/day, and 400 mg/day are also tested in patients following the same protocols and the dose-response relationship is recorded for the extent of virus inhibition.

### Example 6. Treatment of Fibrosis Using ROCK2 Inhibitor

A study is conducted on men and women suspected of having, or having, SARS-CoV-2 infections and suspected of having, or having, fibrosis in the same way as described in Example 1. In addition to testing the extent of virus infection, respiratory symptoms, pulmonary function tests (PFTs), and/or high-resolution computed tomography (HRCT) are performed on the patient at selected intervals such as, for example, daily, every two, three, four, or five days, weekly, bi-weekly, or monthly, to monitor the progression of fibrosis. X-ray test and/or CT scans can be conducted if clinically approved. Further, the period of time over which Compound 1 is administered can be extended in this study beyond two weeks at various dosage levels.

To assess pulmonary function of each of the subjects following treatment with Compound 1, the subjects are evaluated using a forced vital capacity (FVC) test. The Forced Expiratory Volume in One Second (FEV1), the amount of air that is forcefully exhaled in the first second of the FVC test, are measured for each subject either on day 1, day 3, day 7, day 14, monthly, or bi-monthly of the treatment.

### Example 7. Treatment of Fibrosis Using ROCK2 Inhibitor and Remdesivir

A study is conducted of 50 men and women suspected of having, or having, SARS-CoV-2 infections and suspected of having, or having, fibrosis in the same way as described in Example 2. In addition to testing the extent of virus infection, respiratory symptoms, pulmonary function tests (PFTs), and/or high-resolution computed tomography (HRCT) are performed on the patient at selected intervals such as, for example, daily, every two, three, four, or five days, weekly, bi-weekly, or monthly, to monitor the progression of fibrosis. X-ray test and/or CT scans can be conducted if clinically approved. Further, the period of time over which Compound 1 is administered can be extended in this study beyond two weeks at various dosage levels.

To assess pulmonary function of each of the subjects following treatment with Compound 1, the subjects are evaluated using a forced vital capacity (FVC) test. The Forced Expiratory Volume in One Second (FEV1), the amount of air that is forcefully exhaled in the first second of the FVC test, are measured for each subject either on day 1, day 3, day 7, day 14, monthly, or bi-monthly of the treatment.

### Example 8. Treatment of Kawasaki-Like Disease Using ROCK2 Inhibitor

A study is conducted on pediatric patients suspected of having, or having, SARS-CoV-2 infections and suspected of having, or having, Kawasaki-like disease or displaying pediatric multisystem inflammatory syndrome (PMIS) or pediatric inflammatory multisystem syndrome temporally associated with SARS-CoV-2 (PIMS-TS). The study employs Compound 1, which is orally administered 100 mg/day, 200 mg/day, 300 mg/day, and 400 mg/day. Various amounts of Compound 1 are administered at the designated intervals.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3 mL of either viral transport medium (VTM), Amies transport medium, or sterile saline, unless using a point-of-care test for coronaviruses. The sample is tested using a CDC authorized testing method. Different dosages of Compound 1 administered 50 mg/day, 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, or 800 mg/day are also tested in patients following the same protocols and the dose-response relationship is recorded for the extent of virus inhibition.

In addition to testing the extent of virus infection, vasculitis tests, such as, for example, blood tests (for C-reactive protein, complete blood cell count, amounts of anti-neutrophil cytoplasmic antibodies, or other biomarkers), urine test (for red blood cells or protein contents), and/or imaging tests (e.g., X-rays, ultrasound, computerized tomography (CT), magnetic resonance imaging (MRI) and positron emission tomography (PET)) are performed on the patient at selected intervals such as, for example, daily, every two, three, four, or five days, weekly, bi-weekly, or monthly, to monitor the progression of the Kawasaki-like disease, PMIS or PIMS-TS. Further, period of time over which Compound 1 is administered can be extended in this study beyond two weeks at various dosage levels.

To assess the development of the disease, two-dimensional echocardiography is performed on patients to evaluate coronary artery lesions (CAL) at one month of illness. The measurement of each patient includes the diameter of the left main coronary artery (LMCA), the left anterior descending artery (LAD), the left circumflex coronary artery (LCX), and the proximal and middle segments of the right coronary artery (RCA). Z score of each coronary artery is calculated. See Journal of the American Society of Echocardiography, 2011, 24(1). CAL can be defined as z is least 2 of any coronary arteries of LMCA, LAD, LCX, and the proximal and middle segment of the RCA.

### Example 9. Treatment of Kawasaki-Like Disease Using ROCK2 Inhibitor and Remdesivir

A study is conducted of 20 pediatric patients suspected of having, or having, SARS-CoV-2 infections and suspected of having, or having, Kawasaki-like disease or displaying pediatric multisystem inflammatory syndrome (PMIS) or pediatric inflammatory multisystem syndrome temporally associated with SARS-CoV-2 (PIMS-TS). Compound 1, which is orally administered at 100-400 mg daily for 2-3 weeks of the treatment. Predetermined amounts of Compound 1 are administered at designated intervals. In parallel for pediatric patients having a body weight between 3.5 kg and 40 kg, remdesivir is administered via intravenous infusion in a total volume of up to 125 mL 0.9% saline over 30 to 120 minutes: a single loading dose of 5 mg/kg on day 1; a daily loading dose of 2.5 mg/kg on days 2-10.

Subjects are observed from about two weeks to about six months. Coronavirus testing is conducted daily, every two days, every three days, or at other intervals during the treatment by collecting samples from a patient with a nasopharyngeal swab, an oropharyngeal swab, a nasal mid-turbinate swab, or an anterior nares swab. The swab is then placed immediately into a sterile transport tube containing about 2-3 mL of either viral transport medium (VTM), Amies transport medium, or sterile saline, unless using a point-of-care test for coronaviruses. The sample is tested using a CDC authorized testing method. Different dosages of Compound 1 administered 50 mg/day, 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, or 800 mg/day are also tested in patients following the same protocols and record the dose-response relationship for the extent of virus inhibition.

In addition to testing the extent of virus infection, vasculitis tests, such as, for example, blood tests (for C-reactive protein, complete blood cell count, amounts of anti-neutrophil cytoplasmic antibodies, or other biomarkers), urine test (for red blood cells or protein contents), and/or imaging tests (e.g., X-rays, ultrasound, computerized tomography (CT), magnetic resonance imaging (MRI) and positron emission tomography (PET)) are performed on the patient at selected intervals such as, for example, daily, every two, three, four, or five days, weekly, bi-weekly, or monthly, to monitor the progression of the Kawasaki-like disease, PMIS or PIMS-TS. Further, the period of time over which Compound 1 is administered can be extended in this study beyond two weeks at various dosage levels. To assess the development of the disease, two-dimensional echocardiography is performed on patients to evaluate coronary artery lesions (CAL) at one month of illness. The measurement of each patient includes the diameter of the left main coronary artery (LMCA), the left anterior descending artery (LAD), the left circumflex coronary artery (LCX), and the proximal and middle segments of the right coronary artery (RCA). Z score of each coronary artery is calculated. See Renee Margossian et al., Journal of the American Society of Echocardiography, 2011, 24(1): 53-59. CAL can be defined as z is least 2 of any coronary arteries of LMCA, LAD, LCX, and the proximal and middle segment of the RCA.

What is claimed:

1. A method for treating a viral infection in a subject in need thereof by administering to the subject a therapeutically effective amount of a ROCK2 inhibitor.

2. A method for preventing a viral infection in a subject at risk for the viral infection by administering to the subject a prophylactically effective amount of a ROCK2 inhibitor.

3. A method of treating Kawasaki disease, PMIS, or PIMS-TS associated with a viral infection in a subject in need thereof, comprising administering to the patient a therapeutically effective amount of a ROCK2 inhibitor.

4. A method for the treatment or prevention of sequelae resulting from a viral infection in a subject in need thereof, comprising administering to the patient a therapeutically effective amount of a ROCK2 inhibitor.

5. The method of any one of claims 1-4, wherein the ROCK2 inhibitor is a ROCK2 selective inhibitor.

6. The method of any one of claims 1-5, wherein the ROCK2 inhibitor is a ribonucleic acid (RNA).

7. The method of claim 6, wherein the ROCK2 inhibitor is an antisense RNA, a small interfering RNA or a microRNA.

8. The method of any one of claims 1-5, wherein the ROCK2 inhibitor is a compound having the structure of Formula I: 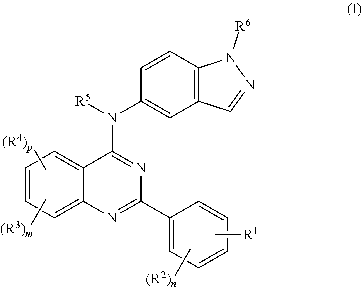
or a pharmaceutically acceptable salt, ester, stereoisomer, polymorph, solvate, N-oxide, or isotopically labeled compound thereof, wherein:
R¹ is selected from the group consisting of O—(CH₂)ᵣCO₂R¹², —O—(CH₂)_{y}, —C(═O)NR¹³R¹⁴, —O—(CH₂)_{y}, -heteroaryl, —O—(CH₂)_{y}, -cycloalkyl, —O—C(═O)—(CH₂)_{y}, —NR¹³R¹⁴, —O—(CH₂)_{z}—NR¹³R¹⁴, NH—C(═O) (CH₂)_{y}, —NR¹³R¹⁴, —NH—C(═O)—X—R¹⁵, and —NH—(CH₂)_{y}, —NR¹³R¹⁴;
R¹² is selected from the group consisting of C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, —(C₁-C₆ alkyl)-O—(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl;
R¹³ and R¹⁴ are independently selected from the group consisting of H, C₁-C₈alkyl, C₂-C₈ alkenyl, C₂-C₈ alkynyl, —(C₁-C₆ alkyl)—O—(C₁-C₆ alkyl), —(C₁-C₆ alkyl)-NR¹⁶R¹⁷, —(C₁-C₆ alkyl)-C(═O)NR¹⁶R¹⁷ aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
X is absent or selected from a —O, NH, and C₁-C₆ alkyl;
R¹⁵ is selected from the group consisting of heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁵ is selected from —(C₁-C₆ alkyl)—O—(C₁-C₆ alkyl), —(C₁-C₆ alkyl)-NR¹⁶R¹⁷, —CO₂R¹⁸, —O—(CH₂)ₓ—CO₂R¹, and C(═O)NR¹⁶R¹⁷;
R¹⁶ and R¹⁷ are independently selected from the group consisting of H, C₁-C₈ alkyl, C₂-C₈ alkenyl, C₂-C₈ alkynyl, —(C₁-C₆ alkyl)—O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R¹⁸ is selected from the group consisting of H, aryl, aralkyl, heteroaryl, C₁-C₆ alkyl, (C₁-C₆ alkyl)—O—(C₁-C₆ alkyl), —(C₁-C₆ alkyl)-NR¹⁶R¹⁷, —(C₁-C₆ alkyl)—O—(C₁-C₆ alkyl)—O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
x is selected from 0 to 6;
y is selected from 0 to 6;
z is selected from 2 to 6;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
R⁴ is selected from H, —(CH₂)ₐ—NR⁴³R⁴⁴, —Y—R⁴², —O—(CH₂)ₐ—CO₂R⁴², —O—(CH₂)ₐ—C(═O)NR⁴³R⁴⁴, —O—(CH₂)ₐ-heteroaryl, —O—(CH₂)ₐ-cycloalkyl, —O—C(═O)—(CH₂)ₐ—NR⁴³R⁴⁴, —O—(CH₂)_{c}—N⁴³R⁴⁴, —NH—C(═O)—Y—R⁴, —NH—C(═O)—(CH₂)ₐ—NR⁴³R⁴⁴;
R⁴² is selected from the group consisting of C₁-C₆ alkyl, —(C₁-C₆ alkyl)—O—(C₁-C₆ alkyl), —(C₁-C₆ alkyl)-NR⁴⁶R⁴⁷, —(C₁-C₆ alkyl)-C(═O)N⁴⁶R⁴⁷, —(C₁-C₆ alkyl)-O—(C₁—C₆ alkyl)-O—(C₁-C₆ alkyl), each of which may be optionally substituted at one or more carbon atoms by from 1 to 3 substituents independently selected from halo, C₁-C₆ alkoxy, hydroxy, amino, cyano and C₁-C₃ perfluoro alkyl;
R⁴³ and R⁴⁴ are independently selected from the group consisting of H, C₁-C₈ alkyl, C₂-C₈ alkenyl, C₂-C₈ alkynyl, —(C₁-C₆ alkyl)—O—(C₁-C₆ alkyl), —(C₁-C₆ alkyl)-NR⁴⁶R⁴⁷, —(C₁-C₆ alkyl)-C(═O)NR⁴⁶R⁴⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁴³ and R⁴⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
Y is absent or selected from O, NH, and C₁-C₆ alkyl;
R⁴⁵ is selected from the group consisting of H, aryl, —(C₁-C₆ alkyl)-O—(C₁-C₆ alkyl), —(C₁-C₆ alkyl)-NR⁴⁶R⁴⁷, —CO₂R⁴⁸, —O—(CH₂)₆—CO₂R⁴⁸, and —C(═O)NR⁴⁶R⁴⁷;
R⁴⁶ and R⁴⁷ independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁴⁶ and R⁴⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁴⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR⁴⁶R⁴⁷, —(C₁-C₆ alkyl)—O—(C₁—C₆ alkyl)—O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
a is selected from 0 to 6;
b is selected from 0 to 6;
c is selected from 2 to 6;
R⁵ is selected from the group consisting of H, C₁-C₆ alkyl, —(CH₂)_{d}—C(═O)—NR⁵³R⁵⁴, —C(═O)—(CH₂)_{d}—NR⁵³R⁵⁴, and —C(═O)—X—R⁵⁵;
R⁵³ and R⁵⁴ are independently selected from the group consisting of H, C₁-C₈ alkyl, C₂-C₈ alkenyl, C₂-C₈ alkynyl, —(C₁-C₆ alkyl)—O—(C₁-C₆ alkyl), —(C₁-C₆ alkyl)-NR⁵⁶R⁵⁷, —(C₁-C₆ alkyl)-C(═O)NR⁵⁶R⁵⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁵³ and R⁵⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁵⁵ is selected from the group consisting of H, aryl, —(C₁-C₆ alkyl)—O—(C₁-C₆ alkyl), —(C₁-C₆ alkyl)-NR⁵⁶R⁵⁷, —CO₂R⁵⁸, —O—(CH₂) CO₂R⁵⁸, and —C(═O)NR⁵⁶R⁵⁷;
R⁵⁶ and R⁵⁷ independently selected from the group consisting of H, C₁-C₈ alkyl, C₂-C₈ alkenyl, C₂-C₈ alkynyl, —(C₁-C₆ alkyl)—O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁵⁶ and R⁵⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁵⁸ is selected from the group consisting of H, aryl, aralkyl, heteroaryl, C₁-C₆ alkyl, —(C₁-C₆ alkyl)—O—(C₁-C₆ alkyl), —(C₁-C₆ alkyl)-NR⁵⁶R⁵⁷, —(C₁-C₆ alkyl)—O—(C₁-C₆ alkyl)—O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
d is selected from 0 to 6;
e is selected from 0 to 6;
R⁶ is selected from the group consisting of H, C₁-C₆ alkyl, (CH₂)ᵣ—C(═O) NR⁶³R⁶⁴, —C(═O)—(CH₂)ᵣ—NR⁶³R⁶⁴, and —C(═O)—X—R⁶⁵;
R⁶³ and R⁶⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁶⁶R⁶⁷, —(C₁-C₆ alkyl)-C(═O)NR⁶⁶R⁶⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁶³ and R⁶⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁶⁵ is selected from the group consisting of H, aryl, —(C₁-C₆ alkyl)-O—(C₁-C₆ alkyl), —(C₁-C₆ alkyl)-NR⁶⁶R⁶⁷, —CO₂R, —O—(CH₂), —CO₂R⁶⁸, and —C(═O)NR⁶⁶R⁶⁷;
R⁶⁶ and R⁶⁷ independently selected from the group consisting of H, C₁-C₅ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁶⁶ and R⁶⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁶⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁶⁶R⁶⁷, —(C₁-C₆ alkyl)—O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
r is selected from 0 to 6;
s is selected from 0 to 6;
n is selected from 0 to 4;
m is selected from 0 to 3; and
p is selected from 0 and 1.

9. The method of claim 8, wherein the ROCK2 inhibitor is a compound having the structure of Formula II: 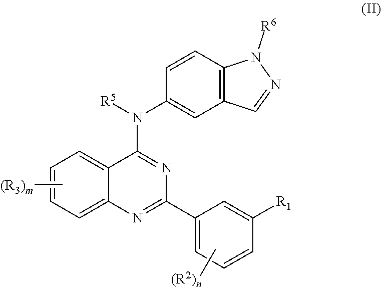
or a pharmaceutically acceptable salt, ester, stereoisomer, polymorph, solvate, N-oxide, or isotopically labeled compound thereof, wherein: R¹, R², R³, R⁵, R⁶, m and n are as defined in claim 8.

10. The method of claim 9, wherein the ROCK2 inhibitor is a compound having the structure of Formula III: 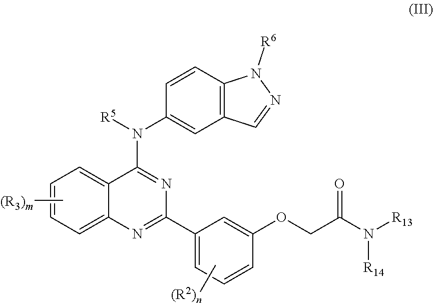
or a pharmaceutically acceptable salt, ester, stereoisomer, polymorph, solvate, N-oxide, or isotopically labeled compound thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)—O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, 3-10 membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form 3-10 membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
wherein each of R², R³, R⁵, R⁶, m and n are as defined in claim 8.

11. The method of claim 10, wherein the ROCK2 inhibitor is a compound having the structure of Formula IV: 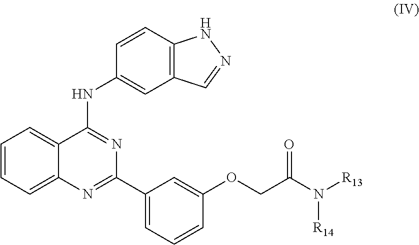
or a pharmaceutically acceptable salt, ester, stereoisomer, polymorph, solvate, N-oxide, or isotopically labeled compound thereof, wherein: R¹³ and R¹⁴ are as defined in claim 10.

12. The method of claim 11, wherein the ROCK2 inhibitor is the compound (2-(3-(4-((1H-indazol-5-yl)amino)quinazolin-2-yl)phenoxy)-N-isopropylacetamide), having the chemical structure of Compound 1: 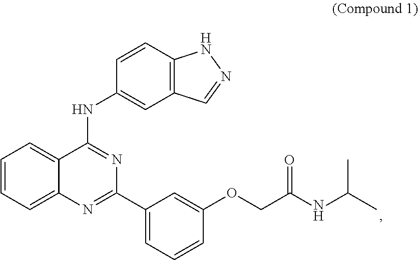
or a pharmaceutically acceptable salt, ester, stereoisomer, polymorph, solvate, N-oxide, or isotopically labeled compound thereof.

13. The method according to any one of claims 1 to 12, wherein the viral infection is caused by a coronavirus.

14. The method according to any one of claims 1 to 13, wherein the viral infection is caused by SARS-CoV-1, SARS-CoV-2 or MERS-CoV.

15. The method according to any one of claims 1 to 14, wherein the viral infection is caused by SARS-CoV-2.

16. The method according to claim 15, wherein the viral infection is caused by a Delta or Omicron variant of SARS-CoV-2.

17. The method according to any one of claims 4 to 16, wherein the sequelae resulting from the viral infection include one or more of the group consisting of fatigue, dyspnea, cough, arthralgia, myalgia, headache, chest pain, fever, palpitations, myocardial inflammation, ventricular dysfunction, stroke, pulmonary function abnormalities, pulmonary fibrosis, renal dysfunction rash, alopecia, olfactory and/or gustatory dysfunction, sleep dysregulation, cognitive impairment altered, memory impairment, depression, anxiety, changes in mood, and combinations thereof.

18. The method according to any one of claims 4 to 17, wherein the sequelae is pulmonary fibrosis.

19. The method according to any one of claims 1 to 18, wherein the method further comprises administering to the subject a therapeutically effective amount of at least one other therapeutic agent.

20. The method according to claim 19, wherein the at least one other therapeutic agent is selected from the group consisting of an antiviral agent, a corticosteroid, an anti-inflammatory signal transduction modulator, a β2-adrenoreceptor agonist bronchodilator, an anticholinergic, a mucolytic agent, hypertonic saline, and combinations thereof.

21. The method according to claim 19, wherein the at least one other therapeutic agent comprises remdesivir.
